(19) United States
(12) Patent Application Publication
(10) Pub. No.: US 2005/0048030 A1
(43) Pub. Date: Mar. 3, 2005

(54) Paramyxoviruses as gene transfer vectors to lung cells

(76) Inventors: Raymond Pickles, Chapel Hill, NC (US); Liqun Zhang, Chapel Hill, NC (US); Mark Peeples, Bexley, OH (US); Peter Collins, Kensington, MD (US); John Olsen, Chapel Hill, NC (US)

Correspondence Address: MYERS BIGEL SIBLEY & SAJOVEC, PO BOX 37428, RALEIGH, NC, 27627, US

(21) Appl. No.: 10/492,733

(22) PCT Filed: Sep. 27, 2002
(86) PCT No.: PCT/US02/30813

Related U.S. Application Data
(60) Provisional application No. 60/326,535, filed on Sep. 28, 2001.

(51) Int. Cl. A61K 48/00; C12N 15/86; C12N 7/00

(52) U.S. Cl. 424/93.2; 435/456; 435/235.1

(57) ABSTRACT

The present invention provides infectious recombinant viral vectors (e.g., parainfluenza virus (PIV) and a respiratory syncytial virus (RSV) vectors) comprising a viral genome comprising a heterologous nucleic acid of interest. Also provided are pseudotyped recombinant viral vectors comprising (i) a viral envelope and (ii) a viral genome comprising heterologous nucleic acids of interest. The viral envelope comprises a structural protein selected from the group consisting of envelope proteins from PIV and/or RSV. Further provided are methods of delivering heterologous nucleic acids of interest into airway epithelial cells comprising introducing viral vectors of the present invention comprising nucleic acids of interest into airway epithelial cells so that the nucleic acids of interest are expressed therein.

### RELATED APPLICATION INFORMATION

This application claims the benefit of U.S. Provisional Application Ser. No. 60/326,535 filed Sep. 28, 2001, which is incorporated by reference herein in its entirety.

### STATEMENT OF FEDERAL SUPPORT

This invention was made with the support of grant number HL 51818-09 from the Heart, Lung and Blood Institute of the National Institutes of Health and with intramural support from the National Institutes of Health. The United States government has certain rights to this invention.

### FIELD OF THE INVENTION

The present invention relates to infectious recombinant viral vectors comprising and capable of expressing nucleic acids and the use of such infectious recombinant viral vectors for transfer of nucleic acids to cells, in particular, airway epithelial cells, and paramyxovirus and paramyxovirus-pseudotyped recombinant viral vectors comprising and capable of expressing nucleic acids.

### BACKGROUND OF THE INVENTION

The Paramyxoviridae include well-known agents that cause disease in both humans and animals. These viruses are enveloped, negative-stranded RNA viruses. The genomic RNA serves as template for synthesis of mRNA and, additionally, as a template for synthesis of the antigenome (+) strand. mRNA synthesis occurs only after the virus has been uncoated and infected host cells, followed by viral replication. During viral replication, the antigenome.(+) strand serves as a template to produce additional copies of the genomic RNA (−) strand.

Human respiratory syncytial virus (RSV) is an important viral agent of serious pediatric respiratory disease worldwide (Collins et al. (2001). In Fields Virology, Fourth Edition, Knipe et al. (eds.) Lippincott, Philadelphia, Pa. vol. 1, p. 1443-1485). RSV infection causes common cold-like symptoms that progress to lower respiratory tract disease in approximately 25-40% of infected infants and results in hospitalization for approximately 0.1 to 1.0% of infected infants. Although most people have been exposed to RSV by two years of age, the immunity induced by RSV infection typically is incomplete and reinfection is common, although subsequent infections are partially restricted and the associated disease is reduced (Collins et al. (2001). In Fields Virology, Fourth Edition, Knipe et al. (eds.) Lippincott, Philadelphia, Pa. vol. 1, p. 1443-1485).

RSV is an enveloped, nonsegmented, negative sense RNA virus classified in subfamily Pneumovirinae of the family Paramyxoviridae, which also includes measles, parainfluenza (PIV) (types 1-4), and mumps virus. When propagated in established cell lines, RSV have been visualized as pleomorphic spheres of 120-300 nm and, more frequently, as long filaments of up to 1-10 μm in length (Collins et al. (2001). In Fields Virology, Fourth Edition, Knipe et al. (eds.) Raven Press, New York, N.Y. p. 1443-1485; Roberts et al. (1995) J. Virol. 69:2667-2673). RSV replicates relatively inefficiently in vitro, and most of the progeny virus remain cell associated (Collins et al. (2001). In Fields Virology, Fourth Edition, Knipe et al. (eds.) Lippincott, Philadelphia, Pa. vol. 1, p. 1443-1485; Levine and Hamilton (1969) Arch. Gesamte Virusforsch. 28:122-132). In contrast, RSV replicates to relatively high titer in the respiratory tract of permissive hosts such as the chimpanzee and humans. Like other members of Paramyxoviridae, RSV gene expression and replication appear to be entirely cytoplasmic, with no apparent direct nuclear involvement. The 15.2 kb RNA genome has been completely sequenced and has been shown to encode 10 mRNAs encoding 11 distinct proteins, and one or more functions have been identified for most of the proteins. Complete infectious recombinant virus has been rescued from plasmids encoding a complete positive-sense copy of the genome together with the proteins of the nucleocapsid/polymerase complex, namely the nucleocapsid N protein, phosphoprotein P, large polymerase protein L, and transcription anti-termination factor M2-1 (Collins et al. (1995) Proc. Natl. Acad. Sci. USA 92:11563-11567).

RSV has three virally-encoded surface proteins: the heavily-glycosylated G protein, which was previously identified as an attachment protein (Levine et al. (1987) J. Gen. Virol. 68:2521-2524); the fusion F protein that mediates membrane fusion at the cell surface resulting in viral penetration; and the SH protein, which does not appear to be necessary for any step in the virus replicative cycle and presently has unknown function. An RSV variant called cp-52 was derived by extensive passage of wild-type virus in vitro and was shown to lack the SH and G genes due to a spontaneous deletion (Karron et al. (1997) Proc. Natl. Acad. Sci. USA 94:13961-13966). Recombinant viruses that have been engineered to lack the G gene, the SH gene, or both also have been derived (Bukreyev et al. (1997) J. Virol. 71:8973-8982; Techaarpornkul et al. (2001) J. Virol. 75:6825-6834). The ability of these G-deletion RSV mutants to efficiently replicate in cultured cells (Karron et al. (1997) Proc. Natl. Acad. Sci. USA 94:13961-13966; Techaarpornkul et al. (2001) J. Virol. 75:6825-6834) indicated that the G protein, like SH, also is dispensable for infection, syncytia formation, and virion morphogenesis, at least in vitro. This suggested that the F protein, the sole remaining viral surface protein, also can act as an attachment protein (Bukreyev et al. (1999) Proc. Natl. Acad. Sci. USA 96:2367-2372; Techaarpornkul et al. (2001) J. Virol. 75:6825-6834).

RSV envelope protein-target cell interaction has been studied predominately in non-polarized epithelial cell lines. Recent studies have suggested that sulfated glycosaminoglycans on the cell membrane are involved in RSV infection (Hallak et al. (2000) Virology 271:264-275; Hallak et al. (2000) J. Virol. 74:10508-10513; Krusat and Streckart (1997) Arch. Virol. 142:1247-1254; Martinez and Melero (2000) J. Gen. Virol. 81:2715-2722). Both the G and F proteins have been shown to bind to cellular glycosaminoglycans (Feldman et al. (2000) J. Virol. 74:6442-6447), consistent with the idea that each protein might function in attachment. In polarized Madin-Darby bovine kidney (MDBK) cell monolayers, RSV infection was shown to result in the budding and release of virus from the apical surface (Roberts et al. (1995) J. Virol. 69:2667-2673).

Human PIV3, a paramyxovirus, contains a single-stranded RNA genome 15.5 kilobases in length. The 3′ and 5′ ends of the viral genome contain extragenic leader and trailer regions that possess promoters required for replication and transcription (Chancock et al. (2001) In Fields Virology, Fourth Edition, Knipe et al. (eds.) Lippincott, Philadelphia, Pa. vol. 1, pp 1341-1380). The genome organization of PIV3 is 3′-leader-N—P(C/D/V)-M-F—HN-L-5′-trailer, and is depicted in FIG. 1. Transcription initiates at the 3′ end and proceeds by a sequential stop-start mechanism that is guided by short conserved motifs found at the gene boundaries. The upstream end of each gene contains a gene-start (GS) signal, which directs initiation of its respective mRNA. The downstream terminus of each gene contains a gene-end (GE) motif, which directs polyadenylation and termination. Each gene is separated by a conserved intergenic trinucleotide. Because of polymerase falloff during sequential transcription, there is a gradient of transcription in which promoter-proximal genes are expressed more efficiently than promoter-distal genes. Viral NP, P, and L proteins, in addition to the viral genome, form the viral nuclear capsid. NP (major nuclear protein) binds to and protects the genome from cellular nucleases. Each NP molecule binds to six nucleotides and only dissociates temporarily during viral replication or transcription. P (phosphor protein) and L (large protein) form viral polymerases, which act on both viral replication and transcription. M (matrix protein) encases viral capsids and mediates viral maturation. The HN (hemagglutinin-neuraminidase) glycoprotein mediates the first step in infection, namely virus adsorption, by attachment to sialic acid, which resides on unknown receptors. The F (fusion) glycoprotein mediates viral penetration of the host cell via fusion of the viral envelope to the plasma membrane.

Giant cell (syncytium) formation leading to cell death is a prominent feature of infection of non-polarized cells with PIV3. This effect is inhibited by treatment of cells with neuraminidase, indicating fusion is highly dependent on its attachment function to sialic acid. Blocking cell fusion prevents the development of cytopathic effects and leads to the establishment of a persistent infection (Moscona and Peluso (1992) J. Virol. 66:6280-6287). In contrast, the pathology of fatal parainfluenza virus disease in human infection usually does not include syncytium formation unless the patient is profoundly immunosuppressed (Weitraub et al. (1987) Arch. Pathol. Lab. Med. 111:569-670). Human infection with PIV3 stimulates both innate immunity (e.g., interferons) and the adaptive immune response, including the development of serum neutralizing antibodies. Consistent with this scenario, children and adults with various forms of immunodeficiency may develop particularly severe illness and can shed virus for a prolonged period (Rabella et al. (1999) Clin. Infect. Dis. 28:1043-1048). Both innate and adaptive immunity are thought to contribute to clearing an infection and to conferring resistance to reinfection. However, protection is short-lived, and resistance associated with serum-neutralizing antibodies is only partial.

The development of the reverse genetics method has made it possible to recover complete infectious recombinant virus entirely from cDNA for a number of paramyxoviruses including RSV (Collins et al. (1995) Proc. Natl. Acad. Sci. USA 92:11563-11567) and PIV3 (Durbin et al. (1997) Virology 235:323-332). In the case of PIV3, this involves cotransfecting cells with plasmids encoding a complete genome or antigenome RNA and the NP, P, and L proteins. Expression of the plasmids, driven by bacteriophage T7 RNA polymerase supplied by a vaccinia virus recombinant or by constitutive expression in an engineered cell line, leads to self-assembly of components into a nucleocapsid, and a subsequent productive infection. Recovered recombinant virus can be propagated as for biologically derived virus.

PIV3 has also been evaluated as a gene transfer vector. For this purpose, a transgene was placed under the control of both gene-start and gene-end PIV3 transcription signals and inserted into the gene boundaries. Since transcription is polarized, transgene placement in a promoter-proximal location provides a higher level of expression. The capacity for accepting additional transgene sequences in a viable recombinant PIV3 is surprisingly large (up to 4 kilobases) with no effect on replication in vitro and modest attenuation in vivo (Skiadopoulos et al. (2000) Virology 272:225-234). A wide variety of attenuated viruses have been created and characterized in the course of developing live attenuated recombinant PIV3 vaccines (Murphy and Collins (2002) J. Clin. Invest. 110:21-27). The fact that a PIV3 vaccine candidate (cp45) can be safely administered to infants and young children (Karron et al. (1995) J. Infect. Dis. 172:1445-1450) suggests that attenuated PIV3 may be useful for constructing safer gene transfer vectors.

Lentiviral vectors have many advantages as gene-transfer vehicles including transduction of nondividing cells, sustained transgene expression from the integrated provirus, and simplicity in modifying tropism by pseudotyping the lentivirus. HIV-based lentiviral vectors (Naldini et al. (1996) Science 272:263-267) pseudotyped with vesicular stomatitis virus envelope glycoprotein (VSV-G) offer the ability to transduce a broad range of different cell types, but fail to transduce differentiated airway epithelia (Goldman et al. (1997) Hum. Gene Ther. 8:2261-2268). Ebola virus pseudotyped HIV vector was found to efficiently transduce airway epithelia (Kobinger et al. (2001) Nat. Biotech. 19:225-230). Interestingly, the authors also reported that HIV vector pseudotyped with RSV envelope proteins (F and G) failed to transduce human airway cells.

Equine infectious anemia virus (EIAV) is a lentivirus that is severely restricted in its host range to horses and closely related equines. EIAV lentiviral vectors (Olsen (1998) Gene Ther. 5:1481-1487) were developed for enhanced safety (as compared to HIV-based vector) for human application. VSV-G pseudotyped EIAV-based lentiviral vectors have been shown to transduce human cells with efficiency similar to that of pseudotyped HIV vectors (O'Rourke et al. (2002) J. Virol. 76:1510-1515).

U.S. Pat. No. 5,962,274 to Parks describes SV5 viral vectors and the administration of such vectors to A549 human lung cells, but such cells are transformed cells and are not differentiated ciliated airway epithelial cells.

Y. Yonemitsu et al. (2000) Nature Biotechnology 18, 970-973 describes the use of Sendai virus to transform mouse and ferret airway cells in vivo, but is not concerned with human cell transformation.

Accordingly, there is a need in this art for gene transfer vectors that can deliver nucleic acids to airway epithelia cells, in particular human ciliated airway epithelial cells in vivo.

### SUMMARY OF THE INVENTION

The present invention may provide improved viral vectors that may be used to introduce nucleic acids of interest into cells such as airway epithelial cells including polarized ciliated airway epithelial cells, and more specifically, human ciliated airway epithelial cells. The viral vectors of this invention are derived from members of the Paramyxoviridae family and include recombinant paramyxovirus vectors and paramyxovirus-pseudotyped virus vectors. In one aspect, this invention provides an infectious recombinant viral vector comprising a viral genome which comprises a heterologous nucleic acid of interest, wherein the viral vector is a parainfluenza virus (PIV) or a respiratory syncytial virus (RSV) vector. In another aspect of this invention, the infectious recombinant RSV or PIV vector comprises a heterologous nucleic acid of interest encoding a cystic fibrosis transmembrane conductance regulator protein (CFTR) or an active fragment of CFTR.

Additional aspects of this invention include a pseudotyped recombinant viral vector comprising (i) a viral envelope and (ii) a viral genome comprising a heterologous nucleic acid of interest, wherein the viral envelope comprises structural proteins such as a PIV F and/or HN protein or an RSV F, SH, and/or G protein.

As still a further aspect, this invention provides a method of delivering a heterologous nucleic acid of interest into a human ciliated airway epithelial cell which comprises introducing a viral vector comprising the nucleic acid of interest into the human ciliated airway epithelial cell so that the nucleic acid of interest is expressed therein. In this particular embodiment, the viral vector is an RSV or PIV vector and the nucleic acid of interest encodes the CFTR protein or an active fragment of CFTR These and other aspects of this invention are set forth in more detail in the following description of the invention.

### BRIEF DESCRIPTION OF THE DRAWINGS

The patent or application file contains at least one drawing executed in color. Copies of this patent or patent application publication with color drawing(s) will be provided by the U.S. Patent and Trademark Office upon request and payment of the necessary fee.

FIG. 1. The genomic organization of recombinant PIV3 viruses. The negative sense RNA genome (3′ to 5′) of wild-type PIV3 is shown on top. Each open box represents a separate encoded polyadenylated mRNA. Above each box are the mRNA length in nucleotides (nt), the length(s) of the encoded protein(s) in amino acids (aa), and the protein name(s); slashes indicate that more than one protein is encoded by a single mRNA. The shaded boxes under the PIV3 genome map represent transgenes (GFP and CFIR as labeled) inserted into the indicated positions of PIV3 genome. The lengths of GFP and CFTR in nt were included in parenthesis. The names of the respective viruses are given below.

FIG. 2. Cell morphology and KS expression at the apical ciliated surfaces of WD HAE cell cultures. (Panel A) Light micrograph of a cross section of a WD HAE culture grown at an ALI on a semipermeable membrane support for 4 weeks. Under these conditions, pseudostratified mucociliary epithelial cell morphology was generated. The cells were counterstained with hematoxylin and eosin. (Panel B) Confocal fluorescent optical section of a live WD HAE culture exposed to an antibody specific for KS and detected with a secondary antibody conjugated to Texas Red. Note that KS serves as a marker for ciliated columnar epithelial cells at the apical surface of the culture and that the permeable support, a 10-μm-deep layer underlying the basal epithelial cells, displays non-KS-specific autofluorescence. Original magnification, ×100.

FIG. 3. Comparison of the abilities of rgRSV and AdVGFP to infect the apical (Ap) versus the basolateral (Bl) surfaces of WD HAE cultures. RgRSV (7×10⁶ pfu; MOI, ˜20) or AdVGFP (10⁸ pfu; MOI, ˜300) was applied to either the apical or basolateral surface of the cultures as detailed in Materials and Methods. Twenty-four hours later, the cultures were analyzed en face for GFP expression by fluorescence photomicroscopy. Original magnification, ×10.

FIG. 4. Polarity of rgRSV infection of WD HAE cultures. Shown are confocal fluorescent-optical-section photomicrographs of HAE cultures inoculated via either the apical (Ap) or basolateral (Bl) surfaces with rgRSV or AdVGFP. Twenty-four hours after infection, the cultures were fixed and immunostained with antibody specific for KS and detected by a secondary antibody conjugated to Texas Red. The KS-expressing apical surfaces of ciliated cells are shown in red, and virus-infected cells are shown in green. Original magnification, ×63.

FIG. 5. Susceptibility of HAE cultures to rgRSV infection as a function of the differentiation state of the culture. (Panel A) Freshly plated cells were grown to confluence to represent a PD cell type and allowed to differentiate with time. On the indicated days following establishment of an ALI, replicate cultures were inoculated with rgRSV (7×10⁶ pfu), and the percentage of GFP-positive cells was quantitated by fluorescence photomicroscopy 24 h later. Each datum point represents the mean of three independent measurements ±standard error of the mean. (Panel B) Representative photomicrographs of the differentiation status of HAE cultures on day 2 (i), day 8 (ii), and day 14 (iii) after initiation of an ALI. Note the abundant ciliated cells on day 14. The cells were counterstained with hematoxylin and eosin. Also shown are en face fluorescence photomicrographs of corresponding cultures expressing GFP 24 h after inoculation with rgRSV on day 2 (iv), day 8 (v), and day 14 (vi). Original magnifications, ×100 (light) and ×10 (fluorescence).

FIG. 6. Polarized release of rgRSV from the apical surfaces of WD HAE cultures. Virus shed from either the apical or basolateral surfaces of six independent cultures was collected at 24 h intervals as described in Materials and Methods. Titration of the collected samples on HEp-2 cells revealed significant shedding of rgRSV from the apical surface (diamonds), whereas within the limits of detection, no viral shedding was measured from the basolateral surface (below limits of detection). The values shown represent the mean ±standard deviation (n=6).

FIG. 7. Spread of rgRSV infection with time in WD HAE cultures. The apical surfaces of cultures were inoculated with a low titer of rgRSV (7×10³ pfu) to achieve a submaximal number of cells expressing GFP at 24 h. Infection was then allowed to proceed over 4 days, and GFP expression was examined en face by fluorescence photomicroscopy on days 1 (Panel A), 2 (Panel B), 3 (Panel C), and 4 (Panel D) postinoculation. Note the counterclockwise circular spread of rgRSV infection by day 2 (Panel B) and the increased number of rgRSV-infected cells by day 4. Original magnification, ×10.

FIG. 8. Inhibition of initial rgRSV infection and spread in WD HAE cultures with an RSV-neutralizing monoclonal antibody or ribavirin. The apical surfaces of HAE cultures were inoculated with rgRSV (10⁵ pfu; MOI, ˜0.3), and GFP expression was monitored en face by fluorescence photomicroscopy 1 (Panel A) and 3 (Panel B) days later. To assess the effects of potential RSV inhibitors on initial rgRSV infection, parallel cultures were treated prior to rgRSV inoculation with either 250 μg of the F-specific RSV-neutralizing monoclonal antibody Synagis/ml applied to the apical surface (Panel C) or 100 μg of ribavirin/ml included in the basolateral medium (Panel D). The cultures were then inoculated with rgRSV as described above, and GFP expression was assessed 1 day later by fluorescence photomicroscopy. To assess the effects of RSV inhibitors on viral spread, parallel cultures were inoculated as described above and then treated with Synagis 6 h postinoculation, and GFP expression was assessed on day 1 (Panel E) and day 3 (Panel F) postinoculation. Cultures treated with ribavirin 24 h postinoculation were assessed for GFP expression by fluorescence photomicroscopy on day 2 (Panel G) and day 4 (Panel H) postinoculation. Original magnification, ×10.

FIG. 9. Cytopathology of different RSV isolates after apical inoculation of WD HAE cultures. The apical surfaces of HAE cultures were inoculated with either rgRSV (10⁶ pfu); GP1, an isogenic recombinant RSV that lacks GFP (10⁶ pfu); HEp-4, a biologically derived wild-type RSV (10⁶ pfu); or the Udorn strain of influenza A virus (10⁶ pfu). The RSV- and influenza virus-inoculated cultures were incubated for 37 and 2 days, respectively. Histological cross sections counterstained with hematoxylin and eosin showed no gross histological differences in cell morphology for the RSV-inoculated cultures compared to cultures not inoculated with any virus. In contrast, cultures inoculated with influenza A virus underwent significant cytopathology 2 days postinoculation. Original magnification, ×63.

FIG. 10. Pseudotyped EIAV lentiviral vector gene transfer to polarized MDCK cells. The apical (Ap) or basolateral (Bl) surface of polarized MDCK cells (Rt>800 Ωcm²) on 0.4 μm T-Col membranes was exposed to an EIAV lacZ vector (UNC-SIN6.1CZW) pseudotyped with VSV-G or the influenza HA, M2, and NA membrane proteins at a MOI of 10. The cultures were stained with X-Gal 96 hours post-transduction.

### DETAILED DESCRIPTION OF THE PREFERRED EMBODIMENTS

The present invention will now be described with reference to the accompanying drawings, in which preferred embodiments of the invention are shown This invention may, however, be embodied in different forms and should not be construed as limited to the embodiments set forth herein Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention. As used in the description of the invention and the appended claims, the singular forms “a”, “an” and “the” are intended to include the plural forms as well, unless the context clearly indicates otherwise.

All publications, patent applications, patents, and other references mentioned herein are incorporated by reference in their entirety.

Nucleotides and amino acids are represented herein in the manner recommended by the IUPAC-IUB Biochemical Nomenclature Commission, or (for amino acids) by either the one-letter code, or the three letter code, both in accordance with 37 CFR §1.822 and established usage. See, e.g., PatentIn User Manual, 99-102 (November 1990) (U.S. Patent and Trademark Office).

Standard techniques for the construction of the vectors of the present invention are well-known to those of ordinary skill in the art and can be found in such references as Sambrook et al. (1989) Molecular Cloning: A Laboratory Manual 2^{nd} Ed. Cold Spring Harbor, N.Y. and F. M. Ausubel et el. (1994) Current Protocols in Molecular Biology Green Publishing Associates, Inc. and John Wiley & Sons, Inc., New York, N.Y. A variety of strategies are available for ligating fragments of DNA, the choice of which depends on the nature of the termini of the DNA fragments and which choices can be readily made by the skilled artisan.

The viral vectors of the present invention may include heterologous (i.e., exogenous) nucleic acids. In embodiments of the invention, the heterologous nucleic acid(s) is expressed within the target cell, and optionally, followed by subsequent production of heterologous proteins or peptides therein.

A nucleic acid or gene sequence is said to be heterologous if it is not naturally present in the wild-type of the viral vector used to deliver the nucleic acid or gene sequence into a cell.

The term “nucleic acid”, “nucleic acid sequence”, or “gene sequence”, as used herein, is intended to refer to a nucleic acid molecule (e.g., DNA or RNA). Such sequences may be derived from a variety of sources including DNA, cDNA, synthetic DNA, RNA, or combinations thereof Such sequences may comprise genomic DNA which may or may not include naturally occurring introns. Genomic or cDNA may be obtained in any number of ways. Genomic DNA can be extracted and purified from suitable cells by means well-known in the art. Alternatively, mRNA can be isolated from a cell and used to prepare cDNA by reverse transcription, or other means well-known in the art.

As used herein, an “isolated” nucleic acid means a nucleic acid separated or substantially free from at least some of the other components of the naturally occurring organism or virus, for example, the cell or viral structural components or other polypeptides or nucleic acids commonly found associated with the nucleic acid.

As used herein, the term “vector” or “gene delivery vector” may refer to a paramyxovirus (e.g., RSV or PIV) particle that functions as a gene delivery vehicle, and which comprises viral genomic RNA (i.e., the vector genome) packaged within a paramyxovirus (e.g., RSV or PIV) envelope. Alternatively, in some contexts, the term “vector” may be used to refer to the vector genomic RNA.

As used herein, a “recombinant paramyxovirus vector genome” is a paramyxovirus genome (i.e., genomic RNA) into which a heterologous (e.g. foreign) nucleotide sequence (e.g., transgene) has been inserted. A “recombinant paramyxovirus particle” comprises a recombinant paramyxovirus vector genome packaged within a paramyxovirus envelope.

Likewise, a “recombinant paramyxovirus vector genome” is a paramyxovirus genome that comprises a heterologous nucleic acid sequence.

Recombinant Paramyxovirus Vectors

The paramyxoviruses used in the practice of the present invention are members of the family Paramyxoviridae (see, e.g., Lamb and Kolakofsky (2001) In Fields Virology, Fourth Edition, Knipe et al. (eds.) Lippincott, Philadelphia, Pa. vol. 1, pp. 1305-1340), and may be from either the subfamily Paramyxovirinae or the subfamily Pneumovirinae. Examples of paramyxoviruses from the subfamily Paramyxovirinae include, but are not limited to, e.g. Sendai virus, parainfluenza viruses, Mumps virus, Newcastle disease virus, and Measles virus. Examples of paramyxoviruses from the subfamily Pneumovirinae include, but are not limited to, e.g. respiratory syncytial viruses, pneumonia virus of mice (PVM), and avian pneumovirus.

The present invention provides vectors derived from paramyxoviruses, e.g., attenuated, recombinant, replication-defective, pseudotyped (as discussed in more detail below) viruses, and the like, as well as viruses that have been modified to express ligands for cell-surface molecules, antibodies or antibodies fragments, and the like.

The paramyxovirus vectors produced according to the present invention are useful for the delivery of nucleic acids to cells in vitro, ex vivo, and in vivo. In particular, the paramyxovirus vectors can be advantageously employed to deliver or transfer nucleic acids to animal, more preferably mammalian, cells. In particular embodiments, and as described in more detail below, nucleic acids of interest include nucleic acids encoding peptides, polypeptides, and proteins, for example, for vaccine or therapeutic purposes (e.g., for medical or veterinary uses). Paramyxoviruses useful in carrying out the present invention are preferably human paramyxoviruses.

The genomic sequences of various paramyxoviruses, as well as the nucleotide sequence of the particular coding regions of the paramyxovirus genomes, are known in the art and may be accessed, e.g., from GenBank. Suitable examples include but are not limited to: respiratory syncytial virus (RSV) strains A2 (GenBank Accession No. M74568), S2 (GenBank Accession No. U39662), and B1 (GenBank Accession No. AF013254); human parainfluenza virus (hPIV) type-1, (GenBank Accession No. AF457102), type-2 (GenBank Accession No. X57559), type-3 (GenBank Accession No. AB012132), measles virus (Genbank Accession No. AB016162) and Newcastle Disease Virus (NDV, GenBank Accession No. AF309418).

Preferred are human PIV type-3 (hPIV3), or RSV. In one particular embodiment, the virus is a human paramyxovirus and is of the subfamily Paramyxovirinae. In another particular embodiment, the virus is a human paramyxovirus and is a member of the subfamily Pneumovirinae.

For purposes of this invention, an “infectious” recombinant viral vector is able to replicate and form new viral particles, i.e., has not had genetic material essential for the replication of the virus deleted or otherwise rendered defective.

As used herein, the term “attenuated virus” refers to any virus of the present invention that has been modified so that its capacity to cause disease or pathology in a host animal or cell is reduced, or even eliminated (i.e., it encompasses viruses that are incapable of causing cytopathic effects in viral cultures, or that are only able to produce reduced cytopathic effects). Thus, the term encompasses viral particles that are capable of some degree of infection and gene expression, but have a reduced ability to produce disease or productive infection. In particular embodiments, an attenuated virus backbone may be used to construct the recombinant vectors wherein a balance between transgene expression and attenuation in viral replication is achieved (e.g., the insertion of a heterologous nucleic acid sequence and, in some instances, the position of the insertion results in attenuation). In other embodiments, the viral genome may be defective for the expression of one or more of the envelope proteins, for example, by deletion of part or all of the sequence(s) encoding the envelope protein(s). In still yet other embodiments, the nucleic acid sequences encoding the envelope protein(s) may be rearranged, recombined, or truncated in such a manner as to lead to attenuation. Attenuated paramyxoviruses are known in the art and include those attenuated viruses described by Collins and Murphy (2002) Virology 296:204-211; Durbin et al. (1999) Virology 261:319-330; Karron et al. (1997) Proc. Natl. Acad. Sci. USA 94:13961-13966; Hansen and Petersen (2002) Curr. Opin. Mol. Ther. 4:324-333; Zuffery et al. (1997) Nat. Bioltechnol. 15:871-874; Arya and Sadaie (1993) J. Acquir. Immune Defic. Syndr. 6:1205-11.

A. Parainfluenza Viruses

Parainfluenza (PIV) viruses which may be used to carry out the present invention include PIV known in the art including, but not limited to, those described in U.S. Pat. No. 6,248,578 to Banerjee et al. Currently, at least four types of human parainfluenza viruses (hPIV) have been recovered. These types include hPIV1, hPVI2, hPVI3 and hPIV4, including subtypes PIV4A and PIV4B (Chancock et al. (2001) In Fields Virology, Fourth Edition, Knipe et al. (eds.) Lippincott, Philadelphia, Pa. vol. 1, pp. 1341-1379).

The wild-type PIV viral genome encodes a major nucleocapsid nuclear protein (NP), a nucleocapsid phosphoprotein (P), a large polymerase protein (L), a matrix protein (M) and a partial or complete genome or antigenome of PIV. The PIV viral genome further encodes support proteins including P/C/D/V, M, F, and HN. At least eight proteins are encoded by PIV3: the nucleocapsid protein N, the phosphoprotein P, the nonstructural protein C, the D protein, the matrix protein M, the fusion glycoprotein F, the hemagglutinin-neuraminidase protein HN, and the large polymerase protein L (Collins et al. (1996) In Fields Virology, Third Edition, Knipe et al. (eds.) Lippincott, Philadelphia, Pa. vol. 1 pp. 1205-1243). The M, HN, and F proteins are envelope-associated, and the latter two are surface glycoproteins which, as is the case with each PIV, are the major neutralization and protective antigens (Collins et al. (1996) In Fields Virology, Third Edition, Knipe et al. (eds.) Lippincoff, Philadelphia, Pa. vol. 1 pp. 1205-1243).

By “PIV antigenome” is meant a positive-sense polynucleotide molecule which serves as the template for the synthesis of progeny PIV genomes. In particular embodiments of the present invention, the genome or antigenome of the recombinant PIV of the invention need only contain those genes or portions thereof necessary to render the viral particles encoded thereby infectious.

The paramyxoviruses of the present invention generally comprise a viral envelope packaging a viral genome (typically, a recombinant viral genome). The envelope comprises at least those proteins necessary for particle assembly and packaging of the viral genome. In particular embodiments, a vector comprising a PIV envelope comprises the PIV F and/or HN protein. In still other embodiments, a vector comprising a PIV3 envelope comprises the PIV3 F and/or HN protein.

The nucleocapsid comprises a vector genome, typically a recombinant vector genome, comprising one or more heterologous nucleic acid sequences (as described below). The vector genome may be replication incompetent in the absence of helper sequences providing in trans the missing functions for the viral genome. For example, the sequence(s) encoding at least one of the envelope proteins may be partially or completely deleted or otherwise mutated so that a functional envelope protein(s) is not produced from the viral genome.

In particular embodiments, the vector is a PIV vector and the heterologous nucleic acid sequence may be inserted downstream from a PIV 3′ promoter. In other particular embodiments, the heterologous nucleic acid sequence is inserted proximal to the promoter (e.g., between the 3′ promoter and the NP gene) into the downstream non-coding region of the viral genome. In still other particular embodiments, the heterologous nucleic acid sequence may be inserted between the NP and P/C/D/V coding regions of the PIV genome. In other particular embodiments, the heterologous nucleic acid sequence may be inserted between the P/C/D/V and M coding regions of the PIV genome. In yet still other particular embodiments, the heterologous nucleic acid sequence may be inserted between the M and HN coding regions of the PIV genome. In still other particular embodiments, the heterologous nucleic acid sequence may be inserted between the HN and L coding regions of the PIV genome. In other particular embodiments, the heterologous nucleic acid is inserted upstream from the 5′ trailer region and downstream from the L coding regions of the PIV genome.

In particular embodiments, the vector is a PIV vector (e.g. a PIV3 vector) and a translational start site of the nucleic acid sequence is preceded upstream by a SacII site. In still other particular embodiments, the vector is a PIV vector and an ATG translational start site is placed upstream of the translational start site of the nucleic acid sequence and in a different reading frame from the translational start site of the nucleic acid sequence such that the expression of the nucleic acid sequence may be reduced compared to expression in the absence of placement of the ATG start site as previously described.

To produce infectious PIV from a cDNA-expressed genome or antigenome, the genome or antigenome is coexpressed with at least those PIV proteins necessary to (i) produce a nucleocapsid capable of RNA replication, and (ii) render progeny nucleocapsids competent for both RNA replication and transcription. Transcription by the genome nucleocapsid provides the other PIV proteins and initiates a productive infection. Alternatively, additional PIV proteins needed for a productive infection can be supplied by coexpression.

Infectious PIV of the invention may be produced by intracellular or cell-free coexpression of one or more isolated polynucleotide molecules (e.g., RNA or DNA) that encode a PIV genome or antigenome RNA, together with one or more polynucleotides encoding at least those viral proteins necessary to generate a transcribing, replicating nucleocapsid. The viral structural and nonstructural proteins may be provided by helper nucleic acid constructs (e.g., plasmids or viral constructs) and may be RNA or DNA, or may alternatively be provided by a stably transformed packaging cell.

In some embodiments of the invention the genome or antigenome of a recombinant PIV (rPIV) need only contain those genes or portions thereof necessary to render the viral particles encoded thereby infectious, i.e., the PIV genome or antigenome encodes all functions necessary for viral growth, replication, and infection without the participation of a helper virus or viral function provided by a plasmid or helper cell line. In other embodiments, the rPIV genome or antigenome is replication-defective in that at least one of the genes encoding one of the envelope proteins necessary for viral replication is partially or entirely deleted or otherwise inactivated so that a functional envelope protein is not expressed from the viral genome or antigenome and, further, the virus unable to produce new viral particles in the absence of trans-complementing sequences. In particular embodiments, F and HN are present. In other particular embodiments, F is present and HN is optionally present. In still other particular embodiments, HN is present and F is optionally present

B. Respiratory Syncytial Viruses

Respiratory syncytial viruses (RSV) which may be used to carry out the present invention include any RSV known in the art including, but not limited to, those described in U.S. Pat. No. 6,284,254 to Murphy et al.; U.S. Pat. No. 6,264,957 to Collins et al.; U.S. Pat. No. 6,077,514 to Maasab et al.; U.S. Pat. No. 5,932,222 to Randolph et al.; U.S. Pat. No. 5,922,326 to Murphy et al.; etc. Moreover, there are at least two subgroups of RSV—subgroup A and subgroup B. RSV strain A2 and RSV strain S2 are within subgroup A, and RSV strain B1 is within subgroup B. (Collins et al. (2001) In Fields Virology, Fourth Edition, Knipe et al. (eds.) Lippincott, Philadelphia, Pa. vol. 1, pp. 1452-1453). RSV strains from either subgroup may be used to carry out the invention. In particular embodiments, RSV strain A2 is used to carry out the present invention.

The RSV viral genome encodes a major nucleocapsid nuclear protein (N), a nucleocapsid phosphoprotein (P), a matrix protein (M), a transcription antitermination factor (M2-1), a large polymerase protein (L), and a partial or complete genome or antigenome of RSV. The RSV viral genome further encodes support proteins selected from the group consisting of NS1, NS2, SH, G, and M2-2.

By “RSV antigenome” is meant a positive-sense polynucleotide molecule, which serves as the template for the synthesis of the negative-sense genome.

In particular embodiments of the present invention, the genome or antigenome of the recombinant RSV of the invention need only contain those genes or portions thereof necessary to render the viral particles encoded thereby infectious. Further, the genes or portions thereof may be provided by more than one polynucleotide molecule, i.e., a gene may be provided by complementation or the like from a separate nucleotide molecule, or can be expressed directly from the genome or antigenome cDNA.

The paramyxoviruses of the present invention generally comprise a viral envelope packaging a viral genome (typically, a recombinant viral genome). The envelope comprises at least those proteins necessary for particle assembly and packaging of the viral genome. In particular embodiments, the virus comprises an envelope comprising RSV F, SH and/or G proteins. In another embodiment, the vector comprises an envelope comprising the RSV F and/or G proteins.

The nucleocapsid comprises a vector genome, typically a recombinant vector genome, comprising one or more heterologous nucleic acid sequences (as described below). The vector genome may be replication incompetent in the absence of helper sequences providing in trans the missing functions for the viral genome. For example, the sequence(s) encoding at least one of the envelope proteins may be partially or completely deleted or otherwise mutated so that a functional envelope protein(s) is not produced from the viral genome.

In particular embodiments, the vector is an RSV vector and the heterologous nucleic acid sequence may be inserted downstream from an RSV 3′ promoter. In other particular embodiments, the heterologous nucleic acid sequence is inserted proximal to the promoter (e.g., between the 3′ promoter and the NS1 gene) into the downstream non-coding region of the viral genome. In still other particular embodiments, the heterologous nucleic acid sequence may be inserted between the NS1 and NS2, NS2 and N, and N and P coding regions of the RSV genome. In other particular embodiments, the heterologous nucleic acid sequence may be inserted between the P and SH coding regions of the RSV genome. In yet still other particular embodiments, the heterologous nucleic acid sequence may be inserted between the SH and F coding regions of the RSV genome. In still other particular embodiments, the heterologous nucleic acid sequence may be inserted between the F and M2 coding regions of the RSV genome. In other particular embodiments, the heterologous nucleic acid sequence is inserted upstream from the 5′ trailer region and downstream of the L coding region of the RSV genome.

To produce infectious RSV from cDNA-expressed genome or antigenome, the genome or antigenome is coexpressed with at least those RSV proteins necessary to (i) produce a nucleocapsid capable of RNA replication, and (ii) render progeny nucleocapsids competent for both RNA replication and transcription. Transcription by the genome nucleocapsid provides the other RSV proteins and initiates a productive infection. Alternatively, additional RSV proteins needed for a productive infection can be supplied by coexpression.

Infectious RSV of the invention may be produced by intracellular or cell-free coexpression of one or more isolated polynucleotide molecules (e.g., RNA or DNA) that encode a RSV genome or antigenome RNA, together with one or more polynucleotides encoding at least those viral proteins necessary to generate a transcribing, replicating nucleocapsid. The viral structural and nonstructural proteins may be provided by helper nucleic acid constructs (e.g., plasmids or viral constructs) and may be RNA or DNA, or may alternatively be provided by a stably transformed packaging cell.

In some embodiments of the invention, the genome or antigenome of a recombinant RSV (rRSV) need only contain those genes or portions thereof necessary to render the viral particles encoded thereby infectious, i.e., the RSV genome or antigenome encodes all functions necessary for viral growth, replication, and infection without the participation of a helper virus or viral function provided by a plasmid or helper cell line. In other embodiments, the rPIV genome or antigenome is replication-defective in that at least one of the genes encoding one of the envelope proteins necessary for viral replication is partially or entirely deleted or otherwise inactivated so that a functional envelope protein is not expressed from the viral genome or antigenome and, further, the virus unable to produce new viral particles in the absence of trans-complementing sequences. In particular embodiments, F, SH, and G are present. In other embodiments, F and SH are present and G is optionally present. In still other embodiments, F and G are present and SH is optionally present. In yet still other embodiments, F is present and SH and G are optionally present.

Paramyxovirus-Pseudotyped Vectors

The present invention further provides viral vectors pseudotyped with one or more paramyxovirus envelope protein(s). In one particular embodiment, the pseudotyped virus is a lentiviruses (e.g., EIAV). Lentiviral vectors are advantageously able to integrate into the host genome, thus, potentially conferring long-term transgene expression. In other embodiments, pseudotyping with a paramyxovirus envelope protein(s) confers some or all of the tropism of the paramyxovirus on the viral vector (e.g., for ciliated airway epithelial cells, in particular, human ciliated airway epithelial cells).

As used herein, the term “lentivirus” refers to a group (or genus) of retroviruses that give rise to slowly developing disease. Viruses included within this group include HIV (human immunodeficiency virus; including HIV type 1, and HIV type 2) and EIAV, the etiologic agent of the human acquired immunodeficiency syndrome (AIDS); visna-maedi, which causes encephalitis (visna) or pneumonia (maedi) in sheep, the caprine arthritis-encephalitis virus, which causes immune deficiency, arthritis, and encephalopathy in goats; equine infectious anemia virus, which causes autoimmune hemolytic anemia, and encephalopathy in horses; feline immunodeficiency virus (FIV), which causes immune deficiency in cats; bovine immune deficiency virus (BIV), which causes lymphadenopathy, lymphocytosis, and possibly central nervous system infection in cattle; and simian immunodeficiency virus (SIV), which cause immune deficiency and encephalopathy in sub-human primates. Diseases caused by these viruses are characterized by a long incubation period and protracted course. Usually, the viruses latently infect monocytes and macrophages, from which they spread to other cells. HIV, FIV, and SIV also readily infect T lymphocytes (i.e., T-cells). Lentiviruses which may be used to carry out the present invention include but are not limited to those that are described in U.S. Pat. No. 6,165,782 to Naldini et al.; U.S. Pat. No. 6,277,633 to Olsen; U.S. Pat. No. 6,312,683 to Kingsman et al.; U.S. Pat. No. 6,428,953 to Naldini et al.; etc. In particular embodiments, the lentivirus is an Equine Infectious Anemia Virus (EIAV, GenBank Accession No. AF033820). In other particular embodiments, the virus is HV, FIV, BIV, or SIV.

Pseudotyping of lentiviruses is known in the art (see, e.g., Naldini et al. (1996) Science 272:263-267; Zuffery et al. (1997) Nature Biotech. 15:871-875; O'Rourke et al. (2002) J. Virol. 76:1510-1515; U.S. Pat. Nos. 6,165,782 and 6,428,953 to Naldini et al.; U.S. Pat. No. 6,277,633 to Olsen; U.S. Pat. No. 6,312,683 to Kingsman et al.). “Pseudotyping” as used herein, refers to the process of replacing the natural envelope of the lentivirus with a heterologous or partially heterologous envelope. A pseudotyped virus or vector display a heterologous envelope protein(s) encoded by another virus, and the pseudotyped virus may advantageously exhibit the tropism of the complementing virus. The term “tropism” as used herein refers to entry of the virus into the cell, optionally and preferably followed by expression (e.g., transcription and, optionally, translation) of sequences carried by the viral genome in the cell, e.g., for a recombinant virus, expression of the heterologous nucleotide sequences(s).

According to one embodiment of the present invention, a lentivirus vector (e.g., EIAV) is pseudotyped with a paramyxovirus (e.g., PIV or RSV) envelope protein(s), thereby conferring tropism for epithelial cells, in particular airway epithelial cells. In still other embodiments, a lentivirus vector (e.g., EIAV) is pseudotyped with PIV or RSV envelope protein(s), thereby conferring tropism for human airway epithelial cells, in particular human ciliated airway epithelial cells. The pseudotyped virus comprises an EIAV viral genome comprising EIAV sequence elements required for assembly and release of viral particles from the vector, expression of the nucleic acid of interest, and expression of the desired envelope. The heterologous nucleic acids of interest are described above. In a particular embodiment, the pseudotyped lentivirus, is preferably, but not limited to EIAV, and is pseudotyped with any one, combination, or all coat proteins from a paramyxovirus or a pneumovirus, such as PIV, RSV, measles, and/or NDV, preferably PIV and RSV, and more preferably, hPIV3.

In one particular embodiment, a pseudotyped recombinant viral vector comprises a viral envelope comprising a structural protein or a group of structural proteins selected from the group consisting of (PIV) F and/or HN protein, or (RSV) F, SH, and/or G protein. In another embodiment, the structural proteins are PIV F and/or HN protein. In another embodiment, the structural proteins are RSV F and/or SH protein. In another embodiment, the structural proteins are RSV SH and/or G proteins. In yet another embodiment, the structural proteins are RSV F and/or G protein. In one particular embodiment, the genes encoding all of the EIAV proteins, including the envelope proteins, are removed from the expression vector. During viral packaging, gag, pol, and rev are supplied in trans from another plasmid.

In still other embodiments, the structural proteins are a modified PIV F and/or HN protein. A preferred modification is the truncation of the cytoplasmic tails of F (C-terminal) and HN (N-terminal) proteins of PIV3. In yet another embodiment, the N-terminal cytoplasmic tail of HN may be replaced with that of an EIAV envelope protein. Similar modifications may be made to other envelope proteins from RSV, measles, and NDV.

Preparation of paramyxovirus-pseudotyped vectors may be carried out according to any protocol known in the art. In one particular embodiment, one or more helper constructs are provided coding for at least the proteins that carry out replication of the genome, and one or more additional constructs are provided encoding the envelope proteins. The constructs may be any vector known in the art, e.g. a plasmid or virus vector. Alternatively, the vector may be produced in a packaging cell line.

In one embodiment, a pseudotyped recombinant vector packaging system comprises two or more (e.g., two, three, or four) vectors constructs. The first vector comprises a nucleic acid sequence of at least part of the EIAV genome, wherein the vector contains at least one defect in at least one gene encoding an EIAV structural protein, and a defective packaging signal. The second vector comprises a nucleic acid sequence encoding the recombinant vector genome, wherein the recombinant vector genome contains a competent packaging signal, and a heterologous nucleic acid(s). The third and/or fourth vectors comprise a nucleic acid expressing a viral envelope protein(s), and each typically contains a defective packaging signal. The combined expression of the two or more packaging constructs results in the production of an infectious virus particle comprising the virus envelope protein(s) and packaging the recombinant viral genome.

Recombinant Paramyxovirus Vectors

As will be appreciated by one skilled in the art, the nucleotide sequence of the inserted heterologous nucleic acid or gene sequence or sequences may comprise the coding sequence of a desired product such as a suitable biologically active protein or polypeptide, immunogenic or antigenic protein or polypeptide, a therapeutically active protein or polypeptide, a reporter or marker protein, or combinations thereof as described above.

Preferably, the heterologous nucleic acid sequence encodes a therapeutically active (e.g., for medicinal or veterinary use) protein or polypeptide. In one particular embodiment, the heterologous gene sequences encode the cystic fibrosis transmembrane conductance regulator (CFTR) protein or biologically active analogs, active fragments, or derivatives thereof. Active fragments of CPTR or truncated CFTR include, but are not limited to, those described in Ostedgaard LS et al. (2002) CFTR with a partially deleted R domain corrects the cystic fibrosis chloride transport defect in human airway epithelia in vitro and in mouse nasal mucosa in vivo. Proc Natl Acad Sci USA. 99(5):3093-3098; Zhang L et al. (1998) Efficient expression of CFTR function with adeno-associated virus vectors that carry shortened CFTR genes. Proc Natl Acad Sci USA. 95: 10158-10163; and Flotte T R et al. (1993) Expression of the cystic fibrosis transmembrane conductance regulator from a novel adeno-associated virus promoter. J. Biol. Chem. 268: 3781-3790.

In another embodiment, the heterologous gene sequences encode the cystic fibrosis transmembrane conductance regulator (CFTR) protein or biologically active analogs, active fragments, or derivatives thereof in addition to Green Fluorescent Protein. Other examples of desired products include, but are not limited to, α₁-antitrypsin, cytokines (e.g., α-interferon, β-interferon, interferon-γ, interleukin-2, interleukin-4, interleukin-10, interleukin-12, granulocyte-macrophage colony stimulating factor, lymphotoxin, and the like), clotting factors (e.g. fibrinogen, prothrombin, tissue thromboplastin, calcium, Factor V, Factor VII, Factor VIII, Factor IX, Factor X, Factor XI, Factor XII, Factor XIII, prekallikrein, high molecular weight kininogen, platelets, etc., particularly Factor VIII and Factor IX), and erythropoietin. Additionally, other agents involved in anti-inflammatory responses may be encoded by the heterologous gene sequence.

Other therapeutic polypeptides include, but are not limited to, dystrophin (including the protein product of dystrophin mini-genes, see, e.g, Vincent et al., (1993) Nature Genetics 5:130), utrophin (Tinsley et al., (1996) Nature 384:349), angiostatin, endostatin, catalase, tyrosine hydroxylase, superoxide dismutase, leptin, the LDL receptor, lipoprotein lipase, ornithine transcarbamylase, β-globin, α-globin, spectrin, α₁-antitrypsin, adenosine deaminase, hypoxanthine guanine phosphoribosyl transferase, β-glucocerebrosidase, sphingomyelinase, lysosomal hexosaminidase, branched-chain keto acid dehydrogenase, RP65 protein, cytokines (e.g., α-interferon, β-interferon, interferon-γ, interleukin-2, interleukin-4, granulocyte-macrophage colony stimulating factor, lymphotoxin, and the like), peptide growth factors and hormones (e.g., somatotropin, insulin, insulin-like growth factors 1 and 2, platelet derived growth factor, epidermal growth factor, fibroblast growth factor, nerve growth factor, neurotrophic factor −3 and −4, brain-derived neurotrophic factor, glial derived growth factor, transforming growth factor −α and −β, and the like), receptors (e.g., the tumor necrosis growth factor receptor), monoclonal antibodies (including single chain monoclonal antibodies; an exemplary Mab is the herceptin Mab). Other illustrative heterologous nucleotide sequences encode suicide gene products (e.g., thymidine kinase, cytosine deaminase, diphtheria toxin, and tumor necrosis factor), proteins conferring resistance to a drug used in cancer therapy, tumor suppressor gene products (e.g., p53, Rb, Wt-1), and any other polypeptide that has a therapeutic effect in a subject in need thereof.

The present invention further finds use in methods of producing antibodies in vivo for passive immunization techniques. According to this embodiment, a paramyxovirus vector expressing an immunogen of interest is administered to a subject, as described herein by direct administration or ex vivo cell manipulation techniques. The antibody may then be collected from the subject using routine methods known in the art. The invention further finds use in methods of producing antibodies against an immunogen expressed from a paramyxovirus vector for any other purpose, e.g., for diagnostic purpose or for use in histological techniques.

The inserted heterologous nucleic acid of interest may be a reporter gene sequence or a selectable marker gene sequence. A reporter gene sequence, as used herein, is any gene sequence which, when expressed, results in the production of a protein or polypeptide whose presence or activity can be monitored. Heterologous nucleotide sequences encoding polypeptides include those encoding reporter polypeptides (e.g., an enzyme). Reporter polypeptides are known in the art and include, but are not limited to, GFP, β-galactosidase, alkaline phosphatase, and chloramphenicol acetyltransferase gene. Preferably, the reporter polypeptide is GFP.

Alternatively, the heterologous gene sequence may comprise a sequence complementary to an RNA sequence, such as an antisense RNA sequence, which antisense sequence can be administered to an individual to inhibit expression of a complementary polynucleotide in the cells of the individual.

Alternatively, the nucleic acid of interest may encode an antisense nucleic acid, a ribozyme (e.g., as described in U.S. Pat. No. 5,877,022), RNAs that effect spliceosome-mediated trans-splicing (see, Puttaraju et al, (1999) Nature Biotech. 17:246; U.S. Pat. No. 6,013,487; U.S. Pat. No. 6,083,702), interfering RNAs (RNAi) that mediate gene silencing (see, Sharp et al., (2000) Science 287:2431) or other non-translated RNAs, such as “guide” RNAs (Gorman et al, (1998) Proc. Nat. Acad. Sci. USA 95:4929; U.S. Pat. No. 5,869,248 to Yuan et al), and the like.

An “immunogenic” peptide or protein, or “immunogen” as used herein is any peptide or protein that elicits an immune response in a subject, more preferably, the immunogenic peptide or protein is suitable for providing some degree of protection to a subject against a disease. The present invention may be employed to express an immunogenic peptide or protein in a subject (e.g., for vaccination) or for immunotherapy (e.g., to treat a subject with cancer or tumors).

An immunogenic protein or peptide, or immunogen, may be any protein or peptide suitable for protecting the subject against a disease, including but not limited to microbial, bacterial, protozoal, parasitic, and viral diseases. For example, the immunogen may be an orthomyxovirus immunogen (e.g., an influenza virus immunogen, such as the influenza virus hemagglutinin (HA) surface protein or the influenza virus nucleoprotein gene, or an equine influenza virus immunogen), or a lentivirus immunogen (e.g., an equine infectious anemia virus immunogen, a Simian Immunodeficiency Virus (SIV) immunogen, or a Human Immunodeficiency Virus (HIV) immunogen, such as the HIV or SIV envelope GP160 protein, the HIV or SIV matrix/capsid proteins, and the HIV or SIV gag, pol and env genes products). The immunogen may also be an arenavirus immunogen (e.g., Lassa fever virus immunogen, such as the Lassa fever virus nucleocapsid protein gene and the Lassa fever envelope glycoprotein gene), a poxvirus immunogen (e.g. vaccinia, such as the vaccinia L1 or L8 genes), a flavivirus immunogen (e.g., a yellow fever virus immunogen or a Japanese encephalitis virus immunogen), a filovirus immunogen (e.g., an Ebola virus immunogen, or a Marburg virus immunogen, such as NP and GP genes), a bunyavirus immunogen (e.g., RVFV, CCHF, and SFS viruses), or a coronavirus immunogen (e.g., an infectious human coronavirus immunogen, such as the human coronavirus envelope glycoprotein gene, or a porcine transmissible gastroenteritis virus immunogen, or an avian infectious bronchitis virus immunogen). The immunogen may further be a polio antigen, herpes antigen (e.g. CMV, EBV, HSV antigens) mumps antigen, measles antigen, rubella antigen, diptheria toxin or other diptheria antigen, pertussis antigen, hepatitis (e.g., hepatitis A or hepatitis B) antigen, or any other vaccine antigen known in the art.

The present invention may also be advantageously employed to produce an immune response against chronic or latent infective agents, which typically persist because they fail to elicit a strong immune response in the subject. Illustrative latent or chronic infective agents include, but are not limited to, hepatitis B, hepatitis C, Epstein-Barr Virus, herpes viruses, human immunodeficiency virus, and human papilloma viruses. Paramyxovirus vectors encoding antigens from these infectious agents may be administered to a cell or a subject according to the methods described herein.

Alternatively, the immunogen may be any tumor or cancer cell antigen. Preferably, the tumor or cancer antigen is expressed on the surface of the cancer cell. Exemplary cancer antigens for specific breast cancers are the HER2 and BRCA1 antigens. Other illustrative cancer and tumor cell antigens are described in S. A. Rosenberg, (1999) Immunity 10:281) and include, but are not limited to: MART-1/MelanA, gp100, tyrosinase, TRP-1, TRP-2, MAGE-1, MAGE-3, GAGE-½, BAGE, RAGE, NY-ESO-1, CDK-4, beta-catenin, MUM-1, Caspase-8, KLAA0205, HPVE&, SART-1, PRAME, p15, and p53 antigens.

Preferably, administration of a paramyxovirus vector comprising one or more heterologous nucleotide sequences encoding an immunogen elicits an active immune response in the subject, more preferably a protective immune response.

An “active immune response” or “active immunity” is characterized by “participation of host tissues and cells after an encounter with the immunogen. It involves differentiation and proliferation of immunocompetent cells in lymphoreticular tissues, which lead to synthesis of antibody or the development of cell-mediated reactivity, or both.” (Herbert B. Herscowitz, Immunophysiology: Cell Function and Cellular Interactions in Antibody Formation, in IMMUNOLOGY: BASIC PROCESSES 117 (Joseph A. Bellanti ed., 1985)). Alternatively stated, an active immune response is mounted by the host after exposure to immunogens by infection or by vaccination. Active immunity can be contrasted with passive immunity, which is acquired through the “transfer of preformed substances (antibody, transfer factor, thymic graft, interleukin-2) from an actively immunized host to a non-immune host.” Id.

A “protective” immune response or “protective” immunity as used herein indicates that the immune response confers some benefit to the subject in that it prevents or reduces the incidence of disease. Alternatively, a protective immune response or protective immunity may be useful in the treatment of disease, in particular cancer or tumors (e.g., by causing regression of a cancer or tumor and/or by preventing metastasis and/or by preventing growth of metastatic nodules). The protective effects may be complete or partial, as long as the benefits of the treatment outweigh any disadvantages thereof.

The term “cancer” has its understood meaning in the art, for example, an uncontrolled growth of tissue that has the potential to spread to distant sites of the body (i.e., metastasize). Exemplary cancers include, but are not limited to, leukemias, lymphomas, colon cancer, renal cancer, liver cancer, breast cancer, lung cancer, prostate cancer, ovarian cancer, melanoma, and the like. Preferred are methods of treating and preventing tumor-forming cancers. The term “tumor” is also understood in the art, for example, as an abnormal mass of undifferentiated cells within a multicellular organism. Tumors can be malignant or benign. Preferably, the inventive methods disclosed herein are used to prevent and treat malignant tumors.

Cancer and tumor antigens according to the present invention have been described hereinabove. Paramyxovirus vectors encoding cancer or tumor antigens may be administered in methods of treating cancer or tumors, respectively.

By the terms “treating cancer” or “treatment of cancer”, it is intended that the severity of the cancer is reduced or the cancer is at least partially eliminated. Preferably, these terms indicate that metastasis of the cancer is reduced or at least partially eliminated. It is further preferred that these terms indicate that growth of metastatic nodules (e.g., after surgical removal of a primary tumor) is reduced or at least partially eliminated. By the terms “prevention of cancer” or “preventing cancer” it is intended that the inventive methods at least partially eliminate or reduce the incidence or onset of cancer. Alternatively stated, the present methods slow, control, decrease the likelihood or probability, or delay the onset of cancer in the subject.

Likewise, by the terms “treating tumors” or “treatment of tumors”, it is intended that the severity of the tumor is reduced or the tumor is at least partially eliminated. Preferably, these terms are intended to mean that metastasis of the tumor is reduced or at least partially eliminated. It is also preferred that these terms indicate that growth of metastatic nodules (e.g., after surgical removal of a primary tumor) is reduced or at least partially eliminated. By the terms “prevention of tumors” or “preventing tumors” it is intended that the inventive methods at least partially eliminate or reduce the incidence or onset of tumors. Alternatively stated, the present methods slow, control, decrease the likelihood or probability, or delay the onset of tumors in the subject.

It is known in the art that immune responses may be enhanced by immunomodulatory cytokines (e.g., α-interferon, β-interferon, γ-interferon, ω-interferon, τ-interferon, interleukin-1α, interleulkin-1β, interleukin-2, interleukin-3, interleukin-4, interleukin 5, interleulkin-6, interleukin-7, interleukin-8, interleukin-9, interleukin-10, interleukin-11, interleukin 12, interleukin-13, interleulkin-14, interleukin-18, B cell Growth factor, CD40 Ligand, tumor necrosis factor-α, tumor necrosis factor-β, monocyte chemoattractant protein-1, granulocyte-macrophage colony stimulating factor, and lymphotoxin). Accordingly, in particular embodiments of the invention, immunomodulatory cytokines (preferably, CTL inductive cytokines) are administered to a subject in conjunction with the methods described herein for producing an immune response or providing immunotherapy.

Cytokines may be administered by any method known in the art. Exogehous cytokines may be administered to the subject, or alternatively, a nucleotide sequence encoding a cytokine may be delivered to the subject using a suitable vector, and the cytokine produced in vivo. In preferred embodiments, a paramyxovirus vector encoding a cytokine is used to deliver the cytokine to the subject.

In one particular preferred embodiment, the heterologous gene sequence encodes the cystic fibrosis transmembrane conductance regulator (CFTR) protein or biologically active analogs, active fragments, or derivatives thereof. In another embodiment, the heterologous gene sequences encode the cystic fibrosis transmembrane conductance regulator (CFTR) protein or biologically active analogs, active fragments, or derivatives thereof in addition to Green Fluorescent Protein. Other examples of desired products include, but are not limited to, α₁-antitrypsin, cytokines (e.g., α-interferon, β-interferon, γ-interferon, interleukin-2, interleukin-4, interleukin-10, interleukin-12, granulocyte-macrophage colony stimulating factor, lymphotoxin, and the like), clotting factors (e.g., fibrinogen, prothrombin, tissue thromboplastin, calcium, Factor V, Factor VII, Factor VIII, Factor IX Factor X, Factor XI, Factor XII, Factor XIII, prekallikrein, high molecular weight kininogen, platelets, etc., particularly Factor VIII and Factor IX), and erythropoietin, and other agents involved in anti-inflammatory responses.

It will be understood by those of ordinary skill in the art that the heterologous nucleotide sequence(s) of interest may be operably associated with appropriate control sequences. For example, the heterologous nucleic acid may be operably associated with expression control elements, such as transcription/translation control signals, origins of replication, polyadenylation signals, and internal ribosome entry sites (IRES), promoters, enhancers, and the like. In particular embodiments, more than one heterologous sequence may be present.

A promoter or enhancer element may be operatively associated with the heterologous nucleic acid. Those skilled in the art will appreciate that a variety of promoter/enhancer elements may be used depending on the level and tissue-specific expression desired. An exemplary promoter that may be operatively associated with the nucleic acid of interest is described in Chow Y H et al. (1997) Development of an epithelium-specific expression cassette with human DNA regulatory elements for transgene expression in lung airways. Proc Natl Acad Sci USA. 94: 14695-14700, wherein this promoter may be active in airway epithelial cells, more specifically human ciliated airway epithelial cells.

It is also understood that the promoter/enhancer may be constitutive or inducible, depending on the pattern of expression desired. The promoter/enhancer may be native or foreign and can be a natural or a synthetic sequence. By foreign, it is intended that the transcriptional initiation region is not found in the wild-type host into which the transcriptional initiation region is introduced.

Promoter/enhancer elements that are native to the target cell or subject to be treated are most preferred. Also preferred are promoters/enhancer elements that are native to the heterologous nucleic acid sequence. The promoter/enhancer element is chosen so that it will function in the target cell(s) of interest. Mammalian promoter/enhancer elements are also preferred. The promoter/enhance element may be constitutive or inducible.

Inducible expression control elements are preferred in those applications in which it is desirable to provide regulation over expression of the heterologous nucleic acid sequence(s). Inducible promoters/enhancer elements for gene delivery are preferably tissue-specific promoter/enhancer elements, and include, but are not limited to, lung specific -promoter/enhancer elements. Other inducible promoter/enhancer elements include hormone-inducible and metal-inducible elements. Exemplary inducible promoters/enhancer elements include, but are not limited to, a Tet on/off element, a RU486-inducible promoter, an ecdysone-inducible promoter, a rapamycin-inducible promoter, and a metalothionein promoter.

In embodiments wherein the heterologous nucleic acid sequence(s) will be transcribed and then translated in the target cells, specific initiation signals are generally required for efficient translation of inserted protein coding sequences. These exogenous translational control sequences, which may include the ATG initiation codon and adjacent sequences, can be of a variety of origins, both natural and synthetic. For example, a translational start site of the nucleic acid of interest may be preceded upstream by a SacII site. According to this embodiment, an ATG translational start site is placed upstream of the translational start site of the nucleic acids of interest and in a different reading frame from the translational start site of the nucleic acids of interest. The placement of the ATG start site upstream of the translational start site of the nucleic acids of interest and in a different reading frame from the translational start site of the open reading frame of the nucleic acids of interest may result in reduced expression of the nucleic acids of interest as compared to expression in the absence of placement of the ATG start site as previously described.

Likewise, in embodiments of the invention, the heterologous nucleic acid(s) is operatively associated with suitable paramyxovirus “gene-start” (GS) and “gene-end” (GE) sequences, which sequences are recognized by the viral replication proteins. These transcriptional control elements may flank the heterologous nucleic acid, with the GS sequence will at the 3′ end and the GS sequence at the 5′ end of the heterologous sequences. Paramyxovirus GE and GS sequences are known in the art.

Exemplary PIV GE sequences include 3′ UCCUAAUUUC 5′ (SEQ ID NO:1) or 3′ UCCUNNUUUC 5′ (SEQ ID NO:2). Exemplary PIV GS sequences include 3′ UUCAUUCUUUUU5′ (SEQ ID NO:3), 3′ UUUAUUCUUUWU 5′ (SEQ ID NO:4), 3′ UUUAUUUCCUATUAGUUUUU 5′ (SEQ ID NO:5), UUAAUAUUUUUU 5′ (SEQ ID NO:6) and 3′ UUUAUAUUUUUU 5′ (SEQ ID NO:7). Illustrative RSV GE sequences include 3′ UCAAUNAAUUUUU 5′ (SEQ ID NO:8), and 3′ UCAUUNUUAUUUU 5′ (SEQ ID NO:9). RSV GS sequences include but are not limited to 3′ CCCCGUUUAA 5′ (SEQ ID NO:10) and 3′ CCCCGUUUAU 5′ (SEQ ID NO:11).

Gene Transfer Technology

The methods of the present invention provide a means for delivering heterologous nucleic acids into a broad phylogenetic range of host cells. The vectors, methods, and pharmaceutical formulations of the present invention are additionally useful in a method of administering a protein or peptide to a subject in need of the desired protein or peptide, as a method of treatment or otherwise. In this manner, the protein or peptide may thus be produced in vivo in the subject. The subject may be in need of the protein or peptide because the subject has an alteration or deficiency of the protein or peptide, or because the production of the protein or peptide in the subject may impart some therapeutic effect, as a method of treatment or otherwise, and as explained further below.

In general, the paramyxovirus vectors produced according to the present invention may be employed to deliver any foreign nucleic acid with a biological effect to treat or ameliorate the symptoms associated with disorders related to gene expression. Illustrative disease states include, but are not limited to, cystic fibrosis and other diseases of the lung, and conditions associated with defective or altered CFTR expression in addition to those affecting the airways such as the bile duct, intestines, vas deferens, sweat glands/ducts, pancreatic ducts, and kidneys.

Gene transfer has substantial potential use in understanding and providing therapy for disease states. There are a number of inherited diseases in which defective genes are known and have been cloned. For deficiency state diseases, gene transfer could be used to bring a normal gene into affected tissues for replacement therapy, as well as to create animal models for the disease using antisense mutations. For unbalanced disease states, gene transfer could be used to create a disease state in a model system, which could then be used in efforts to counteract the disease state. Thus, paramyxovirus vectors produced according to the methods of the present invention permit the treatment of genetic diseases. As used herein, a disease state is treated by partially or wholly remedying the defect, deficiency, or imbalance that causes the disease or makes it more severe. The use of site-specific recombination of nucleic sequences to cause mutations or to correct defects is also possible.

The paramyxovirus vectors produced according to the present invention may also be employed to provide an antisense nucleic acid to a cell in vitro or in vivo. Expression of the antisense nucleic acid in the target cell diminishes expression of a particular protein by the cell. Accordingly, antisense nucleic acids may be administered to decrease expression of a particular protein in a subject in need thereof Antisense nucleic acids may also be administered to cells in vitro to regulate cell physiology, e.g., to optimize cell or tissue culture systems. Alternatively, the paramyxovirus vector may encode any other non-translated RNA, as described in more detail hereinabove.

The invention further finds use in in vitro or in vivo systems for producing a recombinant protein or peptide of interest. This embodiment may be practiced to express any polypeptide of interest, including therapeutic proteins or peptides or industrial proteins or peptides (e.g., industrial enzymes).

In one particularly preferred embodiment, the present invention is employed to express an exogenous CFTR protein or active fragment thereof in epithelium, preferably, ciliated respiratory epithelium.

Subjects, Pharmaceutical Formulations, and Modes of Administration

Suitable subjects to be treated according to the present invention include both avian and mammalian subjects, preferably mammalian. Mammals according to the present invention include but are not limited to canine, felines, bovines, caprines, equines, ovines, porcines, rodents, lagomorphs, primates, and the like, and encompass mammals in utero. Canines, felines, bovines, equines and humans are preferred. Illustrative avians according to the present invention include chickens, ducks, turkeys, geese, quail, pheasant, ratites (e.g., ostrich) and domesticated birds (e.g. parrots and canaries), and include birds in ovo. Chickens and turkeys are preferred.

Any mammalian subject in need of being treated according to the present invention is suitable. Human subjects are preferred. Human subjects of both genders and at any stage of development (i.e., neonate, infant, juvenile, adolescent, adult) can be treated according to the present invention. Human subjects afflicted with cystic fibrosis or other respiratory diseases are preferred.

In particular embodiments, the present invention provides a pharmaceutical composition comprising a virus particle of the invention in a pharmaceutically acceptable carrier and/or other medicinal agents, pharmaceutical agents, carriers, adjuvants, diluents, etc. For injection, the carrier will typically be a liquid. For other methods of administration, the carrier may be either solid or liquid. For inhalation administration, the carrier will be respirable, and will preferably be in solid or liquid particulate form. As an injection medium, it is preferred to use water that contains the additives usual for injection solutions, such as stabilizing agents, salts or saline, and/or buffers.

In general, a “physiologically acceptable carrier” is one that is not toxic or unduly detrimental to cells. Exemplary physiologically acceptable carriers include sterile, pyrogen-free water and sterile, pyrogen-free, phosphate buffered saline. physiologically acceptable carriers include pharmaceutically acceptable carriers.

By “pharmaceutically acceptable” it is meant a material that is not biologically or otherwise undesirable, i.e., the material may be administered to a subject without causing any undesirable biological effects. Thus, such a pharmaceutical composition may be used, for example, in transfection of a cell ex vivo or in administering a viral particle or cell directly to a subject.

One aspect of the present invention is a method of transferring a nucleotide sequence to a cell in vitro. The virus particles may be added to the cells at the appropriate multiplicity of infection according to standard transduction methods appropriate for the particular target cells. Titers of virus to administer can vary, depending upon the target cell type and number, and the particular virus vector, and can be determined by those of skill in the art without undue experimentation. Preferably, at least about 10³ infectious units, more preferably at least about 10⁵ infectious units, are administered to the cell.

The cell(s) to be administered the paramyxovirus vector may be of any type, including but not limited to neural cells (including cells of the peripheral and central nervous systems, in particular, brain cells such as neurons and oligodendricytes), lung cells, cells of the eye (including retinal cells, retinal pigment epithelium, and corneal cells), epithelial cells (e.g., gut and respiratory epithelial cells), muscle cells, dendritic cells, pancreatic cells (including islet cells), hepatic cells, myocardial cells, bone cells (e.g., bone marrow stem cells), hematopoietic stem cells, spleen cells, keratinocytes, fibroblasts, endothelial cells, cells of the bile duct, prostate cells, cells of the vas deferens, cells of the sweat glands/ducts, germ cells, and the like. Alternatively, the cell may be any progenitor cell. As a further alternative, the cell can be a stem cell (e.g., neural stem cell, liver stem cell). As still a further alternative, the cell may be a cancer or tumor cell. Moreover, the cells can be from any species of origin, as indicated above.

The paramyxovirus vectors may be administered to cells in vitro for the purpose of administering the modified cell to a subject. In particular embodiments, the cells have been removed from a subject, the paramyxovirus vector is introduced therein, and the cells are then replaced back into the subject. Methods of removing cells from subject for treatment ex vivo, followed by introduction back into the subject are known in the art (see, e.g., U.S. Pat. No. 5,399,346; the disclosure of which is incorporated herein in its entirety). Alternatively, the recombinant paramyxovirus vector is introduced into cells from another subject, into cultured cells, or into cells from any other suitable source, and the cells are administered to a subject in need thereof.

Suitable cells for ex vivo gene therapy are as described above. Dosages of the cells to administer to a subject will vary upon the age, condition and species of the subject, the type of cell, the nucleic acid being expressed by the cell, the mode of administration, and the like. Typically, at least about 10² to about 10⁸, preferably about 10³ to about 10⁶ cells, will be administered per dose in a pharmaceutically acceptable carrier. The cells transduced with the paramyxovirus or lentivirus vector are preferably administered to the subject in a therapeutically effective amount in combination with a pharmaceutical carrier.

A “therapeutically effective” amount as used herein is an amount that provides sufficient expression of the heterologous nucleotide sequence delivered by the vector to provide some improvement or benefit to the subject. Alternatively stated, a “therapeutically effective” amount is an amount that will provide some alleviation, mitigation, or decrease in at least one clinical symptom in the subject. Those skilled in the art will appreciate that the therapeutic effects need not be complete or curative, as long as some benefit is provided to the subject.

A further aspect of the invention is a method of treating subjects in vivo with the paramyxovirus vector. Administration of the paramyxovirus vector produced according to the present invention to a human subject or an animal in need thereof can be by any means known in the art for administering virus vectors. Compositions including pharmaceutical compositions of the present invention may be prepared as described in U.S. Pat. No. 5,962,274 to Parks directed to compositions comprising viral vectors derived from the paramyxovirus, simian virus 5 (SV5). Preferably, the paramyxovirus vector is delivered in a therapeutically effective dose in a pharmaceutically acceptable carrier.

Dosages of the paramyxovirus vector to be administered to a subject will depend upon the mode of administration, the disease or condition to be treated, the individual subject's condition, the particular virus vector, and the nucleic acid to be delivered, and can be determined in a routine manner. Exemplary doses for achieving therapeutic effects are virus titers of at least about 10⁵, 10⁶, 10⁷, 10⁸, 10⁹, 10¹⁰, 10¹¹, 10¹², 10¹³, 10¹⁴, 10¹⁵ transducing units or more, preferably about 10⁸-10¹³ transducing units, yet more preferably 10¹² transducing units.

In particular embodiments, more than one administration (e.g. two, three, four or more administrations) may be employed to achieve the desired level of gene expression.

Exemplary modes of administration include oral, rectal, transmucosal, topical, transdermal, in utero (or in ovo), inhalation, parenteral (e.g., intravenous; subcutaneous, intradermal, intramuscular, and intraarticular) administration, and the like, as well as direct tissue or organ injection, alternatively, intrathecal, direct intramuscular, intraventricular, intravenous, intraperitoneal, intranasal, or intraocular injections. Injectables can be prepared in conventional forms, either as liquid solutions or suspensions, solid forms suitable for solution or suspension in liquid prior to injection, or as emulsions. Alternatively, one may administer the virus in a local rather than systemic manner, for example, in a depot or sustained-release formulation.

The paramyxovirus vector administered to the subject may transduce any permissive cell or tissue. Suitable cells for transduction by the paramyxovirus vectors are as described above. In particular embodiments, the vectors of the present invention transduce airway epithelial cells. In other embodiments, the vectors of the present invention transduce ciliated airway epithelial cells. In still other embodiments, the vectors of the present invention transduce ciliated airway epithelial cells from the apical surface. In still yet other embodiments, the vectors of the present invention transduce human ciliated airway epithelial cells from the apical surface.

Active compounds of the present invention can be administered to a subject in need thereof by any suitable means including oral, rectal, transmucosal, topical or intestinal administration; parenteral delivery, including intramuscular, subcutaneous, intramedullary injections, as well as intrathecal, direct intraventricular, intravenous, intraperitoneal, intranasal, or intraocular injections. Alternately, one may administer the compound in a local rather than systemic manner, for example, in a depot or sustained release formulation. Administration to the lungs is preferred.

In particular embodiments, active compounds disclosed herein may be administered to the lungs of a subject by any suitable means, but are preferably administered by administering an aerosol suspension of respirable particles comprised of the active compound, which the subject inhales. The respirable particles may be liquid or solid. Aerosols of liquid particles comprising the active compound may be produced by any suitable means, such as with a pressure-driven aerosol nebulizer or an ultrasonic nebulizer. See, e.g., U.S. Pat. No. 4,501,729. Nebulizers are commercially available devices which transform solutions or suspensions of the active ingredient into a therapeutic aerosol mist either by means of acceleration of compressed gas, typically air or oxygen, through a narrow venturi orifice or by means of ultrasonic agitation. Suitable formulations for use in nebulizers consist of the active ingredient in a liquid carrier, the active ingredient comprising up to 40% w/w of the formulation, but preferably less than 20% w/w. The carrier is typically water (and most preferably sterile, pyrogen-free water) or a dilute aqueous alcoholic solution, preferably made isotonic with body fluids by the addition of, for example, sodium chloride.

Optional additives include preservatives if the formulation is not made sterile, for example, methyl hydroxybenzoate, antioxidants, flavoring agents, volatile oils, buffering agents and surfactants.

Aerosols of solid particles comprising the active compound may likewise be produced with any solid particulate medicament aerosol generator. Aerosol generators for administering solid particulate medicaments to a subject produce particles which are respirable, as explained above, and generate a volume of aerosol containing a predetermined metered dose of a medicament at a rate suitable for human administration. One illustrative type of solid particulate aerosol generator is an insufflator. Suitable formulations for administration by insufflation include finely comminuted powders which may be delivered by means of an insufflator or taken into the nasal cavity in the manner of a snuff. In the insufflator, the powder (e.g., a metered dose thereof effective to carry out the treatments described herein) is contained in capsules or cartridges, typically made of gelatin or plastic, which are either pierced or opened in situ and the powder delivered by air drawn through the device upon inhalation or by means of a manually-operated pump. The powder employed in the insufflator consists either solely of the active ingredient or of a powder blend comprising the active ingredient, a suitable powder diluent, such as lactose, and an optional surfactant. The active ingredient typically comprises from 0.1 to 100 w/w of the formulation. A second type of illustrative aerosol generator comprises a metered dose inhaler. Metered dose inhalers are pressurized aerosol dispensers, typically containing a suspension or solution formulation of the active ingredient in a liquified propellant. During use these devices discharge the formulation through a valve adapted to deliver a metered volume, typically from 10 to 150 l, to produce a fine particle spray containing the active ingredient. Suitable propellants include certain chlorofluorocarbon compounds, for example, dichlorodifluoromethane, trichlorofluoromethane, dichlorotetrafluoroethane and mixtures thereof. The formulation may additionally contain one or more co-solvents, for example, ethanol, surfactants, such as oleic acid or sorbitan trioleate, antioxidants and suitable flavoring agents.

The aerosol, whether formed from solid or liquid particles, may be produced by the aerosol generator at a rate of from about 10 to 150 liters per minute, more preferably from about 30 to 150 liters per minute, and most preferably about 60 liters per minute. Aerosols containing greater amounts of medicament may be administered more rapidly.

The dosage of the active compounds disclosed herein or pharmaceutically acceptable salt thereof, will vary depending on the condition being treated and the state of the subject, but generally may be an amount sufficient to achieve dissolved concentrations of active compound on the airway surfaces of the subject of from about 10⁻⁷ to about 10⁻³ Moles/liter, and more preferably from about 10⁻⁶ to about 3×10⁻⁴ Moles/liter. Depending upon the solubility of the particular formulation of active compound administered, the daily dose may be divided among one or several unit dose administrations. Other compounds may be administered concurrently with the active compounds, or salts thereof, of the present invention.

Solid or liquid particulate pharmaceutical formulations containing active agents of the present invention should include particles of respirable size: that is, particles of a size sufficiently small to pass through the mouth and larynx upon inhalation and into the bronchi and alveoli of the lungs. In general, particles ranging from about 1 to 5 microns in size (more particularly, less than about 4.7 microns in size) are respirable. Particles of non-respirable size which are included in the aerosol tend to be deposited in the throat and swallowed, and the quantity of non-respirable particles in the aerosol is preferably minimized. For nasal administration, a particle size in the range of 10-500 m is preferred to ensure retention in the nasal cavity.

In administering the active compounds of the present invention, they may be administered separately (either concurrently or sequentially) or, alternatively and preferably, they may be pre-mixed and administered as preformed conjugates. As an illustrative example, as suitable dose of a transfer vector carrying a heterologous nucleic acid of interest, can be pre-mixed with a targeting molecule (i.e., a bispecific bridging antibody, a peptide, biotin-UTP, etc.) and the complex administered to the subject.

In the manufacture of a formulation according to the invention, active agents or the physiologically acceptable salts or free bases thereof are typically admixed with, inter alia, an acceptable carrier. The carrier must, of course, be acceptable in the sense of being compatible with any other ingredients in the formulation and must not be deleterious to the patient. The carrier may be a solid or a liquid, or both, and is preferably formulated with the compound as a unit-dose formulation, for example, a capsule, which may contain from 0.5% to 99% by weight of the active compound. One or more active compounds may be incorporated in the formulations of the invention, which formulations may be prepared by any of the well-known techniques of pharmacy consisting essentially of admixing the components.

Compositions containing respirable dry particles of active compound may be prepared by grinding the active compound with a mortar and pestle, and then passing the micronized composition through a 400 mesh screen to break up or separate out large agglomerates.

The pharmaceutical composition may optionally contain a dispersant which serves to facilitate the formation of an aerosol. A suitable dispersant is lactose, which may be blended with the benzamil or phenamil in any suitable ratio (e.g., a 1 to 1 ratio by weight).

The present invention further finds use in gene transfer strategies for both the clinical and experimental delivery of genes to the airway epithelium. For example, the present invention may be employed to genetically manipulate cells (e.g. airway cells) in vivo to produce models of disease. In addition, the invention finds use in clinical applications addressing disorders of the lung airway epithelium, e.g., cystic fibrosis. Genetic correction of these cells may also be useful in diseases that result in severe airway inflammation, e.g., α₁-antitrypsin deficiency, asthma and other related diseases.

Likewise, vectors of the invention may be employed to deliver any foreign nucleic acid with a biological effect to treat or ameliorate the symptoms associated with any other disorder related to gene expression. Illustrative disease states include, but are not limited to: cystic fibrosis (and other diseases of the lung), hemophilia A, hemophilia B, thalassemia, anemia and other blood disorders, AIDs, Alzheimer's disease, Parkinson's disease, Huntington's disease, amyotrophic lateral sclerosis, epilepsy, and other neurological disorders, cancer, diabetes mellitus, muscular dystrophies (e.g., Duchenne, Becker), Gaucher's disease, Hurler's disease, adenosine deaminase deficiency, glycogen storage diseases and other metabolic defects, retinal degenerative diseases (and other diseases of the eye), diseases of solid organs (e.g., brain, liver, kidney, heart), and the like.

The present invention is explained in greater detail in the following non-limiting Examples.

### EXAMPLE 1

### Respiratory Syncytial Virus Infects Ciliated Cells of Airway Epithelium Via the Lumenal Membrane

In the present study, we have investigated the mechanism of RSV infection using a model of well-differentiated (WD) human airway epithelium (HAE). WD HAE cultures are derived directly from human lung epithelial tissue and, following seeding in vitro, grow to establish a multilayer, polarized, differentiated cell culture that closely resembles the airway epithelium in vivo with regard to morphology and functions including mucus production and ciliary motion (Matsui et al. (1998) J. Clin. Investig. 102:1125-1131; Pickles et al. (1998) J. Virol. 72:6014-6023). RSV infection was performed using a recombinant RSV that expresses green fluorescent protein (rgRSV), providing the means to directly visualize infected cells (Techaarpornkul et al. (2001) J. Virol. 75:6825-6834). This showed that RSV preferentially targets the ciliated cells of the airway epithelium, and that infection (and subsequent virus release) occurs exclusively via the apical surface. In addition, RSV infection persists in this tissue model for greater than three months without obvious cytopathic effects whereas, in contrast, infection with influenza A virus results in rapid and extensive cytopathology.

A. Materials and Methods

Viruses. The construction of rgRSV (224) has been described in detail elsewhere (Hallak et al. (2000) J. Virol. 74:10508-10513). Briefly, GFP (Life Technologies, Gaithersburg, Md.) was engineered to be flanked by RSV gene start and gene end sequences and was inserted as the first, promoter-proximal gene in a full-length cDNA of the wild-type RSV strain A2 antigenomic RNA. RgRSV was rescued by cotransfecting HEp-2 cells with the antigenomic plasmid and N, P, M2-1, and L support plasmids and infecting them with a modified vaccinia virus, MVA-T7, expressing T7 RNA polymerase (Wyatt et al. (1995) Virology 210:202-205). Virus stocks were prepared in HEp-2 cells and were aliquoted and stored at −80° C. until use. For HEp-2 cells, rgRSV was found to replicate to near-parental titers and to produce syncytia at a rate similar to that of the parental virus. Recombinant wild-type RSV without GFP (GP1) and a biologically derived RSV strain (HEp-4) were also used in this study. The Udorn strain of influenza A virus (A/Udorn/72) was provided by Brian Murphy (National Institute of Allergy and Infectious Disease) and was propagated in HEp-2 cells in the presence of 0.75 μg of trypsin/ml. Nonreplicating adenoviral vectors expressing GFP (AdVGFP) were obtained from the University of North Carolina Gene Therapy Vector Core Facility.

Viruses released into the apical compartment were harvested by adding 200 μl of medium to the apical surface of the culture for 20 min and, after retrieval with a pipette, combining it with an equal volume of 2× viral stabilizing solution (200 mM MgSO₄, 100 mM HEPES, pH 7.5), snap freezing the mixture on dry ice, and storing it at −80° C. Two hundred microliters of basolateral medium from a total of 1 ml was retrieved and combined with an equal volume of 2× viral stabilizing solution as described above. Viral titration was performed as described previously and corrected for differences in sample volume (Murphy et al. (1990) Vaccine 8:497-502).

WD HAE cell culture. Human nasal, tracheobronchial, and bronchiolar airway epithelial cells were obtained from cystic fibrosis (CF) patients and non-CF patients undergoing surgical procedures, and epithelial cells were isolated by the University of North Carolina Cystic Fibrosis Center Tissue Culture Core Facility using Institutional Review Board-approved protocols. Following enzymatic dispersion, cells were seeded on collagen-coated, semipermeable membrane supports (Transwell-Col; 12 mm in diameter; 0.4-μm pore size; Corning-Costar, Corning, N.Y.) as previously described (Pickles et al. (1998) J. Virol. 72:6014-6023). At confluence, the apical medium was removed and the cells were maintained at an air-liquid interface (ALI) to allow differentiation of the epithelial subtypes. WD cultures, identified as cultures containing ciliated cells and with transepithelial resistances of ≧300 ωcm², were studied approximately 4 to 6 weeks after initiation of an ALI unless otherwise stated. In some cases, primary cells were further expanded on tissue culture dishes before being seeded onto Transwells (passage 1 cells). Both primary and passage 1 cultures derived from non-CF and CF sources were used in the study and showed no significant differences in any of the parameters tested.

Viral inoculation of HAE cultures. Frozen aliquots of rgRSV or AdVGFP were thawed immediately before use and diluted in tissue culture medium. After the apical surfaces of HAE cultures were rinsed with medium, 100 μl of viral suspension was applied to the apical surface for 1 h at 37° C., and the virus was removed by washing with medium. Inoculation of the basolateral surface of the cultures was performed by inverting the insert and exposing the permeable support to a volume and a concentration of virus equal to those used for the apical inoculation.

For experiments with RSV antibody and ribavirin, the reagents were diluted in tissue culture medium immediately before use. Antibody (250 μg/ml) was applied to the apical or basolateral surfaces of cultures at the time of rgRSV inoculation or as otherwise noted. Ribavirin was added to the basolateral medium at a final concentration of 100 μg/ml.

photomicrographs of GFP-expressing cells were acquired using a Leica Leitz DM IRB fluorescence inverted microscope equipped with a Hamamatsu C5810 color 3 chilled charge-coupled device digital camera and Adobe photoshop. Quantitation of infected cells was performed with the image-processing toolkit plug-ins for photoshop (ISBN 1-928808-00-X).

Immunolocalization of ciliated-cell-specific KS. Keratan sulfate (KS) immunolocalization was performed with HAE cultures fixed with 4% paraformaldehyde. The apical surfaces of cultures were directly exposed to 10% normal goat serum to block nonspecific attachment prior to addition of a KS-specific monoclonal mouse immunoglobulin G (IgG) antibody (MAB2022; Chemicon, Temecula, Calif.), followed by goat anti-mouse IgG-conjugated to Texas Red (Jackson ImmunoResearch, West Grove, Pa.). Texas Red fluorescence was recorded by optical-sections using confocal laser scanning microscopy (Leica DM IRBE).

Reagents. Humanized monoclonal antibody directed to the F protein of RSV (Synagis) was obtained as a kind gift from MedImmune Inc. (Gaithersburg, Md.). Ribavirin was purchased from ICN Biochemicals Inc. (Aurora, Ohio). All other reagents and chemicals, unless otherwise noted, were obtained from Sigma Chemical Company (St. Louis, Mo.).

B. Results

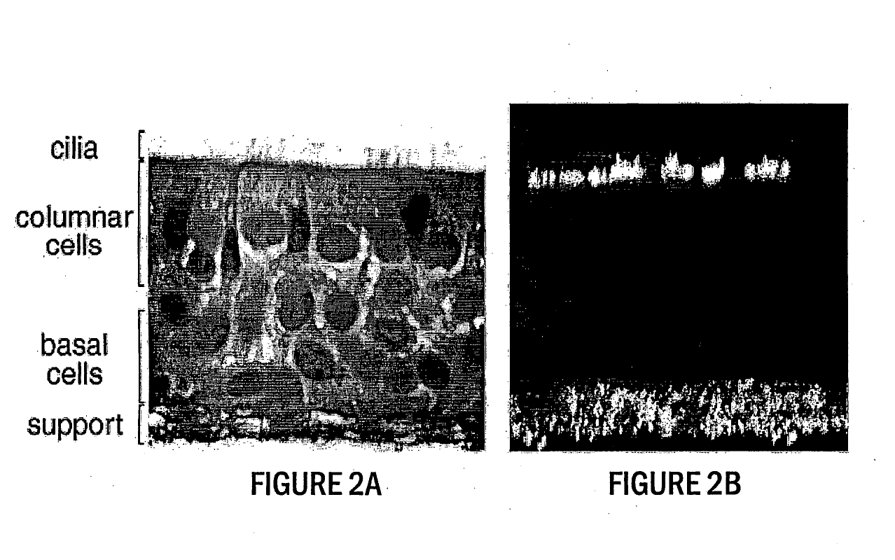

Polarity of rgRSV infection in HAE cultures. We previously used rgRSV to monitor infection of HEp-2 cell monolayers, in particular to characterize the involvement of cell surface glycosaminoglycans in virus attachment and infection in vitro (Hallak et al. (2000) Virology 271:264-275; Hallak et al. (2000) J. Virol. 74:10508-10513). In the present study, we used rgRSV to monitor infection of WD HAE cultures. These cultures are polarized and pseudostratified, with mucociliary cells at the apical surface, similar in both morphology and cell type distribution to the respiratory epithelium in vivo. The pseudostratified mucociliary epithelial cultures are composed of a number of different cell types, including lumen-facing ciliated cells, mucus-secreting cells, and intermediate and basal cell types in the basolateral compartment. A light photomicrograph (FIG. 2, Panel A) of a cross section of a WD HAE culture depicts the pseudostratified mucociliary epithelium with an abundance of ciliated cells. Approximately 25% of the total cells within a culture are lumenal cells. Confocal fluorescence optical sectioning of cultures probed with antibody to KS followed by a Texas Red-conjugated secondary antibody specifically identified the cilia of ciliated columnar airway epithelial cells (FIG. 2, Panel B).

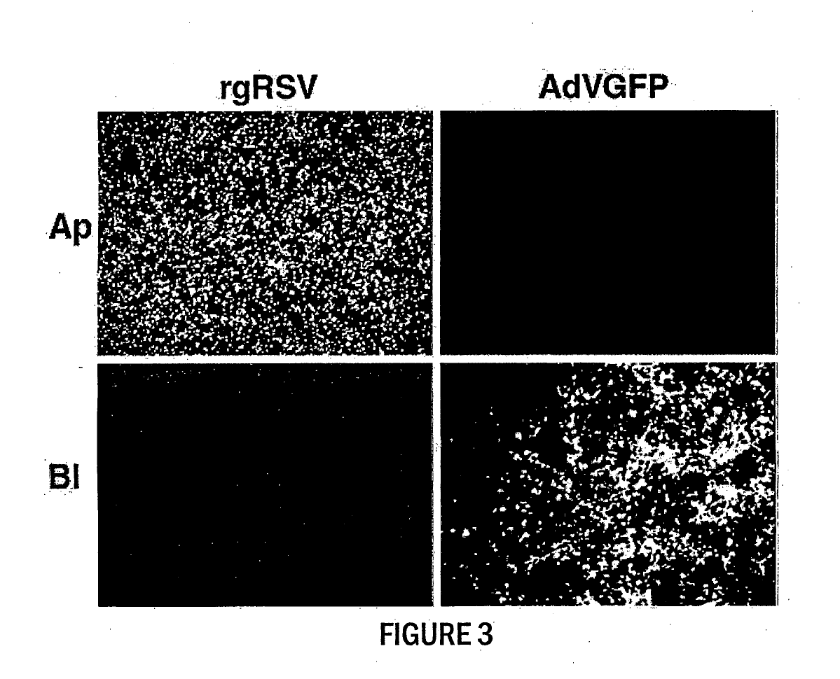

Using this culture system, access to the apical and/or basolateral surface of the epithelium allowed investigation of whether rgRSV can infect the HAE via either surface. HAE cultures were inoculated with rgRSV (7×10⁶ pfu; multiplicity of infection [MOI], ˜20) applied to either the apical or basolateral surface for 1 h, washed, incubated for a further 24 h, and examined by fluorescence microscopy en face to detect expression of GFP as an indication of rgRSV infection. RgRSV infected HAE cells with high efficiency following inoculation of the apical surface, whereas inoculation of the basolateral surface resulted in little or no infection (FIG. 3). In contrast, as previously reported, AdVGFP (10⁸ pfu; MOI, ˜300) applied to the apical surface resulted in no GFP expression, whereas application to the basolateral surface resulted in efficient gene transfer.

Since RSV is pleomorphic and can vary in size, the possibility existed that the pore size of the Transwell-Col membrane support (0.4 μm) might restrict RSV access to the basolateral surfaces of the cultures. To investigate this possibility, we filtered rgRSV through 0.4-μm-pore-size Transwell-Col membrane supports positioned above the apical surfaces of WD HAE cultures. The efficiencies of rgRSV infection were similar whether rgRSV was applied directly to the apical surface or passed through the membrane support (data not shown), indicating that the inability of rgRSV to infect via the basolateral surface was not due to pore size restriction of the membrane support. These results show that rgRSV efficiently infects WD HAE cells via the apical but not the basolateral surface, which is the direct inverse of the polarized gene transfer characteristics of AdV.

For culture preparations from 10 different donors treated with the highest dose of rgRSV (7×10⁶ pfu; MOI, ˜20), a range of infection efficiencies was observed (30 to 80% of cells infected), with an average overall efficiency of ˜52%. These data show that in this model of HAE, rgRSV is able to efficiently infect epithelial cells via the lumenal (apical) membrane, but they suggest that not all of the luminal cells were readily infected.

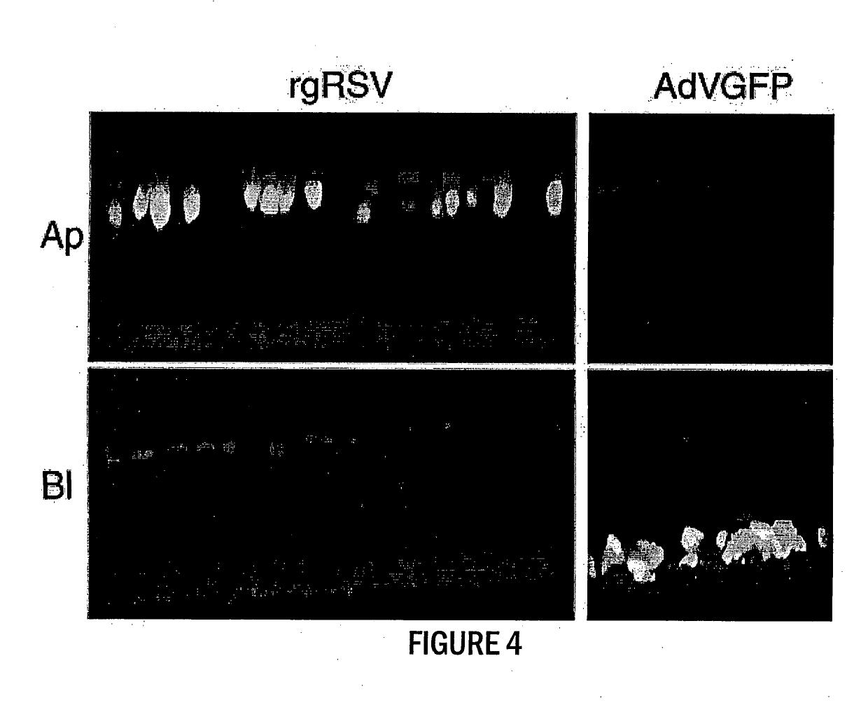

RgRSV specifically infects ciliated cells of the apical surface. In order to identify the cell types infected by rgRSV, HAE cultures were inoculated via the apical or basolateral surface with rgRSV as described above, incubated for 24 h, immunostained for KS, and visualized by confocal optical sectioning. FIG. 4 shows that rgRSV-mediated GFP expression was localized to lumenal-surface columnar cells. Furthermore, the cells infected by rgRSV represented the ciliated subpopulation of lumenal cells. Although not every ciliated cell in a particular culture was infected by rgRSV, probably due to a limitation of rgRSV titer, those that were infected were exclusively ciliated cells. Basolateral inoculation of rgRSV resulted in little or no GFP expression in any cell type within the epithelium (<0.01% of cells). In contrast, parallel studies with AdVGFP revealed an absence of GFP expression following inoculation of the apical surface, whereas efficient expression was observed following basolateral inoculation, with basal cells as the preferential target cell type for AdV, as previously reported (Pickles et al. (1998) J. Virol. 72:6014-6023). These data suggest that, in an intact epithelium, rgRSV preferentially targets ciliated cells of the apical surface.

The results presented above indicated that the cell layer at the basal surface is refractory to rgRSV infection while the ciliated cells of the lumenal surface are readily infected. It was of interest to determine whether other cell types, e.g., intermediate cells, within the multiplayer WD HAE cultures could also be infected with rgRSV. This possibility was evaluated using an epithelium injury model that allows lumenally applied virus to reach underlying intermediate and basal epithelial cells. WD HAE cultures were mechanically injured with a pipette tip, followed immediately by inoculation with either rgRSV or AdVGFP on the apical surface for 1 h. The cultures were then incubated for 24 h, immunostained for KS, and visualized en face. It was observed that rgRSV infection occurred only in intact apical regions of the epithelium, coincident with KS staining, with few GFP-expressing cells present in the region of injury. In contrast, cultures inoculated with AdVGFP were transduced only within the region of injury, where basal cells were exposed (data not shown). Thus, nonlumenal airway epithelial cells (basal and intermediate cells) exposed by mechanical damage were confirmed to lack KS, as expected, and were not susceptible to infection by rgRSV. In contrast, as described previously, HAE cells that underwent mechanical damage were readily infected by AdVGFP.

To further test the effect on rgRSV infection by disturbing the integrity of the lumenal cell layer, the epithelial cell tight junctions were transiently opened by the transient application of EGTA (10 mM) to the apical surface to allow virus access to the basolateral membranes of the cells. This treatment has been shown to produce a significant increase in AdV-mediated gene transfer, since opening the tight junctions allows access of AdV to basolaterally located receptors (Coyne et al. (2000) Am. J. Cell Mol. Biol. 23:602-609; Walters et al. (1999) J. Biol. Chem. 274:10219-10226). No differences were observed in the efficiency of rgRSV infection or in the cell type infected by rgRSV between cultures that maintained intact tight junctions and those in which tight junctions were transiently opened (data not shown). In sum, these results indicate that rgRSV specifically targets the apical surfaces of ciliated airway epithelial cells and that this tropism is not based on physical accessibility required for entry.

We also examined whether cultures generated from proximal and distal airway regions were also susceptible to rgRSV infection. For cultures prepared from nasal, tracheobronchial, and bronchiolar epithelium, rgRSV exhibited the pattern of infecting ciliated lumenal cells (data not shown). These results suggest that all regions of the conducting airway epithelium are susceptible to infection by rgRSV and that at each location rgRSV preferentially, and perhaps exclusively, infects ciliated cells.

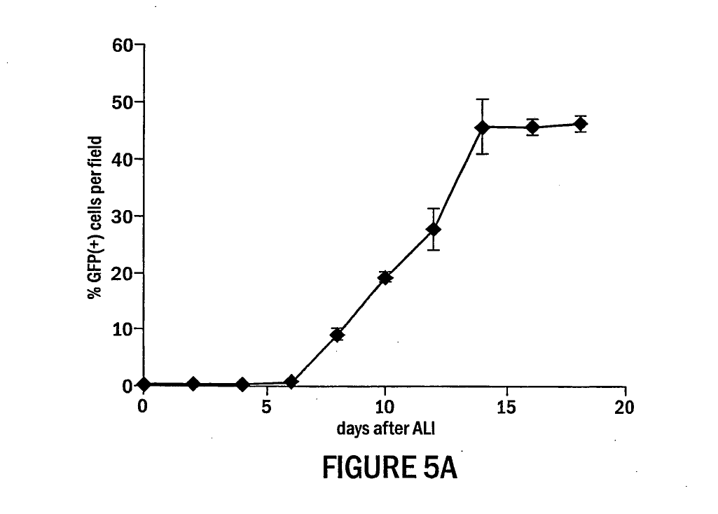

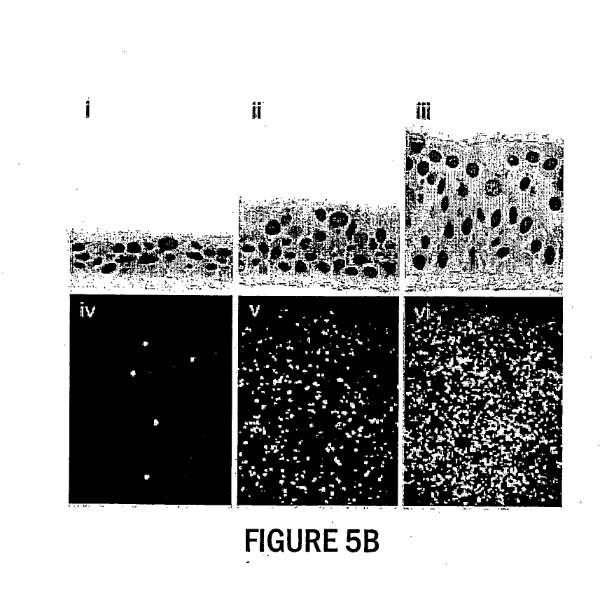

Susceptibility of HAE cultures to rgRSV infection requires differentiation and is coincident with ciliogenesis. We and others have previously shown that the apical surfaces of WD HAE cultures are resistant to AdV-mediated gene transfer because the receptors required for AdV entry are absent from the apical surfaces of airway epithelia (Pickles et al. (1998) J. Virol. 72:6014-6023; Zabner et al. (1997) J. Clin. Investig. 100:1144-1149. However, poorly differentiated (PD) HAE cultures, which are confluent, immature cultures that are precursors to WD HAE, can be transduced by AdV with high efficiency due to the availability of AdV receptors and uptake mechanisms (Pickles et al. (1998) J. Virol. 72:6014-6023). To determine whether the HAE differentiation state affected susceptibility to rgRSV infection, we applied rgRSV to cultures at different stages in the differentiation process under otherwise identical conditions. PD HAE cultures that were inoculated with rgRSV had no evidence of GFP expression 24 to 48 h later (data not shown), indicating that these cells are not susceptible to rgRSV infection. In other cultures, after the establishment of an ALI, susceptibility to apical infection with rgRSV was evaluated as a function of time. As shown in FIG. 5, Panel A, very few cells were susceptible to infection on day 2, whereas susceptibility increased substantially by day 6, reaching a maximum on day 14. Interestingly, susceptibility to infection correlated with ciliogenesis of the columnar cells of the apical surface (FIG. 5, Panel B). These results indicate that rgRSV infection is differentiation dependent and that the extent of infection is directly related to the presence of ciliated columnar epithelial cells.

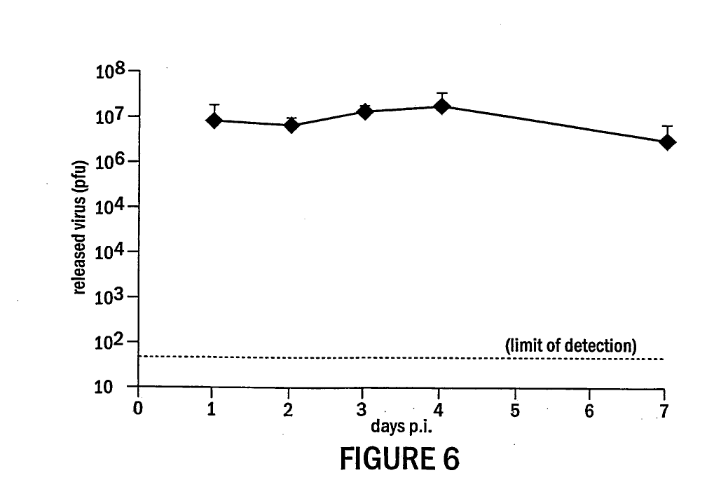

RgRSV infection, spread, and shedding occur at the apical surfaces of HAE cultures. Since rgRSV infects ciliated cells via the apical membrane, we investigated whether rgRSV was shed from the apical and/or basolateral surfaces of WD HAE cultures. The apical surfaces of cultures were inoculated with rgRSV, and infection was allowed to proceed over the following 7 days. On each day postinoculation, samples derived from either the apical or basolateral surfaces were obtained, and the amount of rgRSV in these samples was determined by standard titration on HEp-2 cells. For six individual cultures sampled for 7 days, in all cases rgRSV was shed only from the apical surfaces, as shown in FIG. 6. Within the limits of detection, no rgRSV was shed from the basolateral surfaces of HAE cultures. These data indicate that both the initial infection and subsequent virus shedding for rgRSV are polarized to the apical surfaces of ciliated cells in HAE.

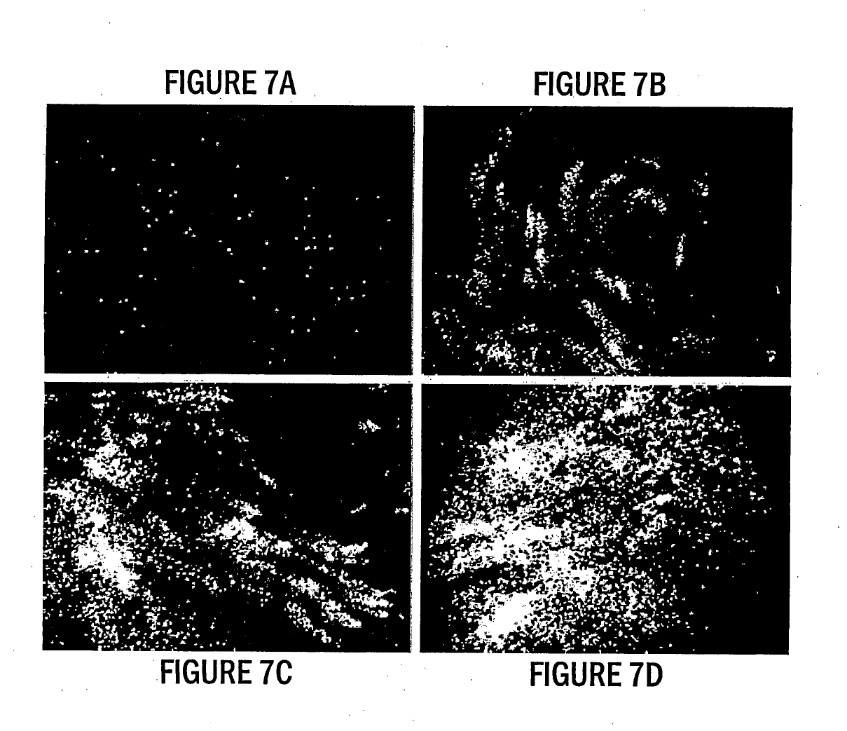

To monitor the time course of rgRSV infection of WD HAE cells, cultures were inoculated at the apical surface with a small amount of virus (7×10³ pfu), and fluorescence photomicrographs were taken en face at 1-day intervals to visualize GFP expression. At 24 h postinoculation, rgRSV infection resulted in a small number of individual green cells, as shown in FIG. 7, Panel A. The spread of rgRSV infection in the cultures over the next 24 h showed a vectorial pattern radiating from each focal infection point, forming a circular pattern of infection (FIG. 7, Panel B). This pattern was consistent with the pattern of ciliary movement in these cultures. Over the next 48 h, rgRSV replication and spread led to a large proportion (>80%) of infected cells (FIG. 7, Panel D).

These observations suggest that rgRSV buds from the apical surface and is released into the lumenal periciliary fluid and/or overlying mucus layer. Thereafter, rgRSV is spread vectorially to adjacent cells within these compartments.

Effects of a neutralizing antibody and ribavirin on rgRSV infection. We further characterized the model by examining the effect of two clinically relevant antiviral agents effective against RSV infection. One agent, Synagis, is a humanized monoclonal antibody specific to the F protein that efficiently neutralizes viral infectivity. This antibody approach is currently in use in passive parenteral immunoprophylaxis in high-risk infants. The second agent, ribavirin, is a nucleoside analog that is used clinically as therapy for RSV infection. We tested the abilities of Synagis and ribavirin to inhibit both initial infection and subsequent spread of rgRSV in WD HAE cultures.

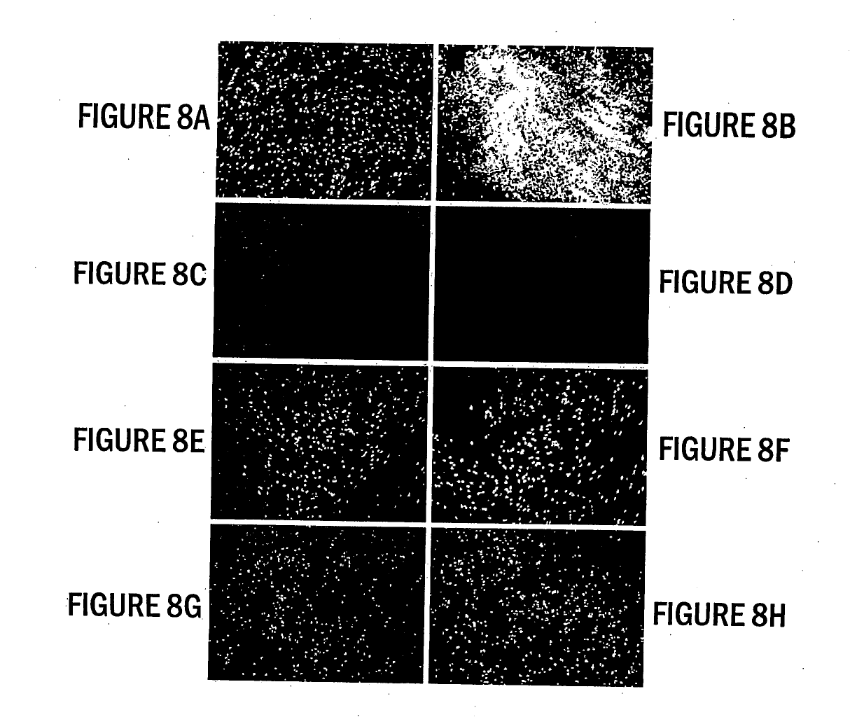

To determine whether these reagents could inhibit initial rgRSV infection, the antibody (250 μg/ml) was mixed with virus and applied to the apical surface, whereas ribavirin (100 μg/ml) was added to the basolateral medium immediately before inoculation. As shown in FIGS. 7, Panels C and D, respectively, both the antibody and the ribavirin treatments resulted in complete inhibition of rgRSV infection compared to the control (FIG. 8, Panel A). To determine whether antibody and ribavirin were also able to reduce viral spread in cultures after infection with rgRSV, either antibody (apical) or ribavirin (basolateral) was applied 6 or 24 h, respectively, after inoculation of WD HAE with rgRSV. As expected, infected cells were detected 24 h postinoculation, but 3 days later there was no evidence of viral spread for cultures that received either the antibody (FIG. 8, Panel F) or ribavirin (FIG. 8, Panel H) treatment, in contrast to the rapid spread of rgRSV in the untreated cultures (FIG. 8, Panel B). Removal of antibody or ribavirin from the respective cultures allowed the resumption of rgRSV spread within 48 h (results not shown).

Additional experiments were performed to determine whether the anti-F antibody inhibited rgRSV infection and spread when the antibody was exposed to the basolateral rather than the apical surface. Antibody applied to the basolateral surface either at the time of or 24 h prior to rgRSV inoculation was not effective in reducing the infection or spread of rgRSV compared to control cultures (results not shown). These data illustrated the efficacy of these clinical strategies to reduce the infectivity of RSV in a model of HAE. In particular, these experiments illustrated that antibody applied to the luminal surface efficiently gained access to the local site of infection so that all viral spread was inhibited. These data are consistent with the efficacy of Synagis given parentally but point to the requirement for antibody to reach apical compartments in order to be effective therapeutically.

Persistence of rgRSV in RAE without obvious cytopathology. In general, the number of cells expressing GFP in WD HAE cultures peaked 2 to 3 days after initial infection, followed by a decrease in the number of positive cells over the next 36 days to a level approximately 25% of that at day 3, at which point the number of infected cells stabilized.

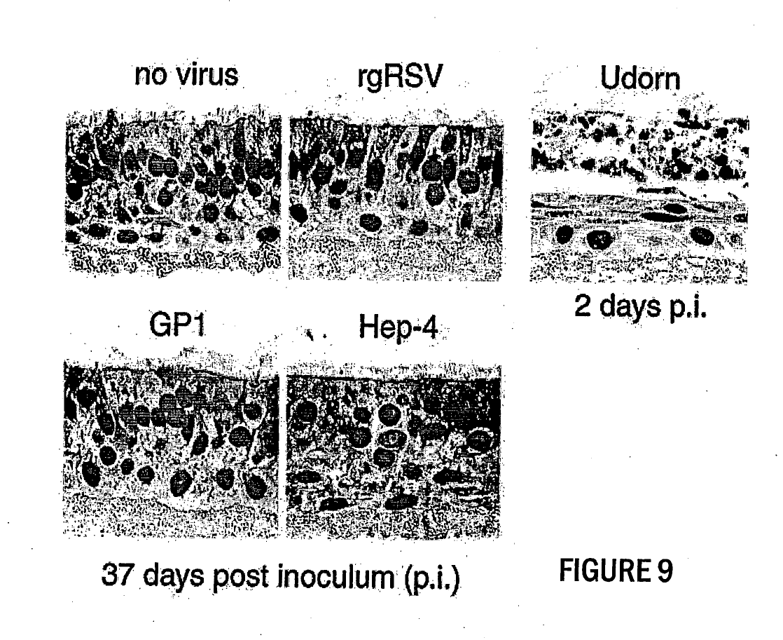

For periods of up to 3 months, the longest interval studied, at the light microscope level, the histological integrity of rgRSV-infected cultures was not detectably altered compared to uninfected cultures from the same source. Specifically, cells appeared to be normal following rgRSV infection, there was no syncytium formation, and the cilial beat was visually unaltered. Histological examination of cultures infected by rgRSV for more than a month revealed no gross histological differences and, importantly, no cell fusion or syncytium formation (data not shown). We also infected WD HAE with a wild-type recombinant RSV that lacks the GFP gene (GP1), the direct parent of rgRSV, and with biologically derived wild-type RSV (HEp-4). Over a period of 36 days, these cultures also failed to display obvious cytopathology (FIG. 9). In contrast, cultures infected with influenza A virus (Udorn strain) exhibited dramatic, rapid destruction and shedding of the columnar airway epithelial cells, as has been observed in vivo (Hers (1966) Am. Rev. Respir. Dis. 93(Suppl.):162-177; Hers and Mulder (1961) Am. Rev. Respir. Dis. 83:84-97; Wright and Webster (2001) In Fields Virology Knipe et al. (eds.), Lippencott Williams and Wilkins, Philadelphia, Pa. p. 1533-1579).

### EXAMPLE 2

### Human Parainfluenza Virus Type 3 Infects Ciliated Cells of Airway Epithelium Via the Lumenal Membrane

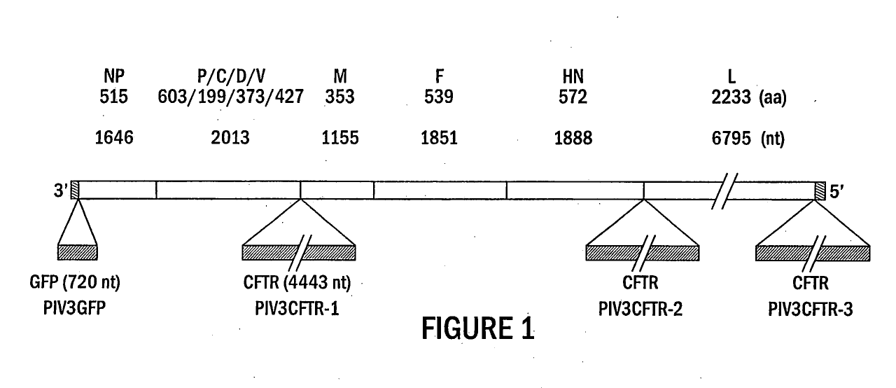

One of the major barriers to the successful delivery of transgenes to the airway epithelium is the inefficiency of vector entry across the apical surface after intralumenal delivery. The ciliated columnar epithelial cells of both the surface epithelium and the submucosal glands are considered to be the cell-type that require correction in CF lung disease and the ability to target CFTR expression in these cell-types is highly desirable. In an attempt to achieve this goal, we have tested a common respiratory pathogen, human parainfluenza virus type 3 (hPIV3) for its ability to infect airway epithelium after intralumenal delivery. A recombinant PIV3 (hPIV3GFP, see FIG. 1 for genome organization) was constructed that expressed a reporter green fluorescent protein (GFP) transgene that allowed for monitoring infection with time. In this study we have used a well-characterized in vitro model of primary human airway epithelial cells (HAE) that recapitulates the morphology and physiology of the human airway epithelium in vivo to test whether hPIV3 can infect HAE by breaching the barrier posed by the apical surface.

To determine the polarity of infection of hPIV3, the apical and basolateral surfaces of HAE were inoculated for one hour with increasing doses of hPIV3GFP (up to 10⁷ pfu/culture; MOI˜30), and GFP expression assessed 24 hours post inoculum. No GFP expression was observed in cultures inoculated via the basolateral surface while there was a dose-dependent increase in the number of GFP-expressing cells after apical surface inoculation with the highest dose of virus resulting in GFP expression in >90% of cells present at the lumenal surface. The results observed for hPIV3GFP (Data not shown) are similar to those observed for rgRSV depicted in FIG. 3.

Laser scanning confocal microscopy was used to generate optical sections of hPIV3GFP infected cultures and revealed that hPIV3GFP infected cells were exclusively lumen-facing ciliated columnar epithelial cells since GFP expression co-localized to cells that also expressed β-Tubulin IV, a protein expressed exclusively in the cilial shaft (Data not shown). The results with hPIV3GFP are similar to those observed for rgRSV shown in FIG. 4, in which GFP expression from rgRSV was observed to co-localize with KS.

In a mechanical injury model that exposed the basolateral surfaces of apical columnar cells as well as the underlying basal/intermediary cell layers, apical inoculation of hPIV3GFP resulted in GFP expression only in the intact, undamaged regions of the cultures similar to that observed for rgRSV, and suggests that hPIV3 preferentially enters ciliated columnar cells through the intact apical membrane.

Pretreatment of the apical surface of HAE with neuraminidase III (NAIII, 160 mU/ml for 3 hrs) followed by hPIV3GFP inoculation resulted in 98% inhibition of GFP expression assessed 24 hours post inoculum, indicating that apical surface sialic acid residues are involved the attachment/entry pathway(s) of hPIV3 into ciliated cells of HAE.

### EXAMPLE 3

### Pseudotyped EIAV Lentiviral Vectors Transduce Polarized MDCK Cells from the Apical Surface

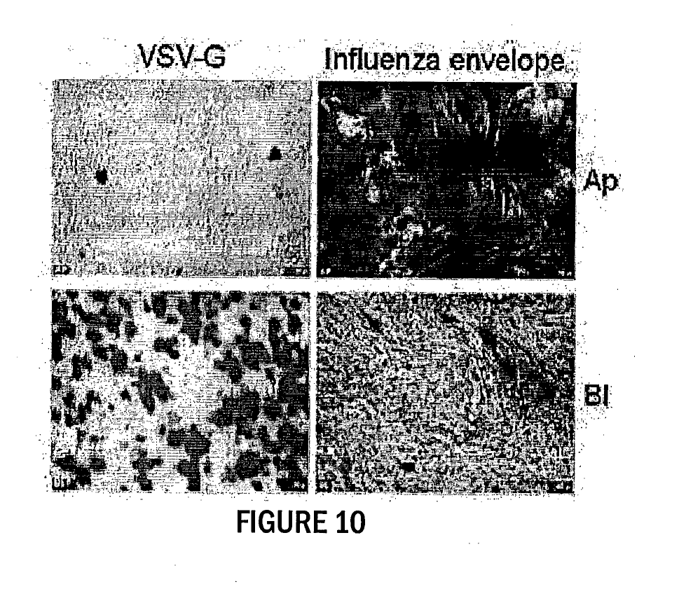

EIAV lentiviral vectors have been successfully pseudotyped with all three membrane proteins, HA, M2, and NA of influenza virus type A. The pseudotyped vector was found to transduce polarized MDCK cells from the apical surface as depicted in FIG. 10. In contrast, EIAV pseudotyped with vesicular stomatitis virus protein G (VSV-G) only transduced from the basolateral surface. Furthermore, the apical transduction of polarized MDCK cells by influenza pseudotyped EIAV vectors was found to be sensitive to neuramidase III treatment, indicating that apical surface sialic acid residues are involved the attachment/entry pathway(s).

### EXAMPLE 4

### The Generation of Recombinant PIV3 Viruses Expressing CFTR (PIV3CFTR)

The coding sequence of CFTR, flanked by the gene-start (10 nt) and gene-end (13 nt) transcription signals of PIV3, is inserted into the downstream end of one of several PIV3 genes. Since CFTR is expressed at a low level in human airways (Trapnell et al. (1991) Proc. Natl. Acad. Sci. USA 88:6565-6569), a relatively low expression level of CFTR transgene is likely desirable, although it is also of interest to obtain recombinants expressing higher levels should that prove feasible. The major factor for determining the level of gene expression by PIV3 is the distance of a gene from its 3′ promoter with promoter-proximal genes being expressed more efficiently than promoter-distal genes. Since the optimal level of CFTR gene expression needed to correct the CF phenotype is unknown, we plan to construct several PIV3 viruses, displaying a gradient of CFTR expression (see FIG. 1 for positions of CFTR insertions). In the first construct, the CFTR expression cassette is inserted between P and M genes (designated PIV3CFTR-1); while in a second, the CFTR is between the HN and L genes (PIV3CFTR-2). In a third construct, CFTR is placed at the end of the genome, after gene L (PIV3CFTR-3). In addition, each construct is designed so that the translational start site of the CFTR gene is preceded shortly upstream-by a unique SacII site. This site can be used to insert a small oligonucleotide duplex that places an ATG translational start site shortly upstream of, and in a separate reading frame from, the translational start site of the CFTR ORF. Based on a large body of published work (Kozak (1986) Cell 44:283-292), this upstream ATG diverts a large fraction of ribosomes into an alternate reading frame, thereby bypassing the CFTR ORF and effecting an anticipated 5- to 10-fold reduction in expression for each construct. The magnitude of this diversion can be controlled to some extent by the nucleotide context of the upstream ATG. The combination of gene position and modified translational start site provides a wide range of expression of CFTR. In addition to testing CFTR expression, the effects of CFTR insertion into the PIV3 genome on attenuation of viral replication in vitro is evaluated.

### EXAMPLE 5

### CFTR Gene Transfer by PIV3 to Airway Epithelium in CF RAE Cultures

CF HAE cultures infected from the apical surface by PIV3CFTR viruses is studied using standard ion transport techniques to determine whether PIV3-mediated CFTR expression is efficient enough to functionally correct the CF bioelectric defect. The effect of increasing doses of PIV3CFTR viruses and the duration of CFTR expression on the efficiency of correction is evaluated.

Primary human airway tracheobronchial epithelial cells are obtained from airway specimens resected at lung transplantation. Primary cells are expanded on plastic to generate passage 1 (P1) cultures. P1 cultures display identical morphology to cultures derived directly from primary cells. P1 cells are plated at 250 k cells on permeable Snapwell-clear (12 mm diameter, Corning) supports for bioelectric measurement or T-Col supports for other studies. Cultures are grown at the air-liquid interface (ALI) to generate well-differentiated, polarized cultures that resemble the pseudostratified mucociliary epithelium that occurs in vivo (Zhang et al. (2002) J. Virol. 76:5654-5666). Well-differentiated cultures, judged by transepithelial resistance (>300 cm²), the presence of ciliated cells, and mucus secretion, are usually obtained four weeks after ALI.

10³-10⁷ pfu of PIV3CFTR viruses diluted in culture media are exposed to the apical membrane of CF cultures for 1 hr. PIV3GFP serves as a negative control for ion transport studies. The first measurement of ion transport is performed at 2 and 3 days pi (when transgene expression is expected to be at the highest level based on data from PIV3GFP infections) as described below, and ion transport measurement continued for at least 14 days.

Western blot analyses and immunofluorescent detection is performed to determine the quantity and localization of CFTR protein in infected CF HAE cultures as for PIV3CFTR producing cells. For immunolocalization, cultures fixed with PFA, dehydrated in serially increasing concentrations of ethanol, and embedded in paraffin, are cut into 5 μm sections and rehydrated. Sections are probed with anti-CFTR antibody, and detected by AlexaFluor-conjugated secondary antibody. Cultures infected with PIV3GFP serve as negative controls. Positive controls for CFTR detection are Calu-3 cells (human lung adenocarcinoma, ATCC) grown on T-Col, which express high level of endogenous CFTR.

Ion transport studies are performed with HAE grown on Snapwells using standardized protocols (Barker et al. (1995) Am. J. Physiol. 268:L270-277). Briefly, the ion transporting capability of HAE is compared at specific time intervals (between 1 and 14 days pi) after PIV3CFTR or PIV3GFP inoculations. A standard pharmacological protocol for assessment of sodium and chloride ion channel activity in epithelial cells is performed. The effects of amiloride (10.4M, sodium conductance blocker), forskolin (10.5 M, cAMP-chloride conductance activator) and ATP (10.4M, calcium-activated chloride conductance) are assessed after sequential exposure of these reagents to the apical surface of HAE. This well-established protocol allows the contribution of the CFTR (cAMP-activated chloride channel) function to be assessed, and so determine the degree of correction of the CF bioelectric defect. In addition, infection by PIV3GFP as monitored by GFP expression shows the percentage of cells infected for a particular code of cultures.

### EXAMPLE 6

### Pseudotyping EIAV Lentiviral Vectors with PIV3 Envelope Proteins

PIV3 envelope glycoproteins F and HN are determinants of PIV3 tropism to human airway ciliated epithelium. EIAV lentiviral vectors pseudotyped with surface glycoproteins HA, AN, and AU of influenza virus type A have been shown to transduce polarized epithelial cells from the apical surface (see Example 3).

Since PIV3 attachment and fusion to target cells can be accounted for with F and HN surface glycoproteins, both F and HN genes are amplified from the viral genome and cloned into mammalian expression vectors, to allow for generation of pseudotyped lentivirus. PIV3 pseudotyped EIAV vectors are produced by a four-plasmid co-transfection of 293 cells (human embryonic kidney) with CaPO₄ precipitation. The four plasmids used for transfection include two plasmids expressing F and HN separately, an EIAV protein (gag-pol-rev) expression plasmid, and a gene transfer plasmid expressing either GFP or CFTR. Stable packaging cell lines that express F and HN of PIV3 in addition to EIAV proteins (except tat and the envelope protein) facilitate production of the pseudotyped vector.

PIV3 genomic RNA is purified from virus-containing media using techniques known in the art. Complementary DNA (cDNA) is synthesized with random decamers as primers (RETROscript Kit, Ambion). PCR primers are designed to flank the coding sequences of PIV3 F and HN genes, with Kozak sequence added at the 5′ ends. PCR-amplified sequences are cloned into pEF6/V5-His-TOPO vector (Invitrogen) for high-level expression in mammalian cells from the human EF-1α promoter. The cloned F and HN are confirmed by sequencing. These two plasmids (pF and pHN) are transiently transfected into 293 cells, and F and HN expression is visualized by immunolabeling with F and HN specific mAbs followed by AlexaFluor conjugated secondary antibody. Toxicity of F and HN is determined individually and combined by scoring syncytium formation.

Either GFP or CFTR is inserted into the gene transfer plasmid pUNC-SIN6.1CW (Patel et al. (2002) Mol. Ther. 5:s171), which contains all cis-acting sequence elements required to support reverse transcription and integration of the vector genome and a multiple cloning site for insertion of cDNAs encoding genes of interest. A low-toxicity GFP (Vitality hrGFP, Stratagene) and human CFTR is inserted into the multiple cloning site of pUNC-SIN6.1CW, to generate pUNC-GFP and pUNC-CFTR. The pEV53B plasmid express EIAV proteins required for assembly and release of viral particles from cells and includes genes encoding proteins from the gag and pol genes as well as the regulatory proteins rev (Patel et al. (2002) Mol. Ther. 5:s171). PIV3 pseudotyped EIAV vectors are produced by CaPO₄-mediated transient transfection of 293 cells in the presence of 10 mM sodium butyrate. The four-plasmid co-transfection at equal amount will include pEV53B gag-pol-rev plasmid, pF and pHN glycoprotein expression plasmids, and pUNC-GFP or pUNC-CFTR reporter plasmid. At 48 hrs after transfection, culture media is harvested, filtered through 0.45 μm filters, and stored at −80° C. until testing. Pseudotyped EIAV vectors are titrated on 293 cells by scoring GFP-expressing cells for EIAV-GFP or by immunostaining with anti-CFTR antibody as described earlier for PIV3CFTR.

An EIAV helper cell line, B241 (Patel et al. (2002) Mol. Ther. 5:s171), was stably transfected with pEV53B, which contains all EIAV encoded proteins except the envelope protein and tat. This colony-purified cell line was found to have stable helper activity and complement virus production following co-transfection with an envelope plasmid and a gene transfer plasmid. B241 is transfected with pcDNA6/TR [which contains the tetracycline repressor (TetR) protein, Invitrogen], and selected with blasticidin containing media, to yield B241TR. B241TR is clone-selected for stable expression of TetR. To generate inducible F and HN expression plasmids, F and HN cDNA is shuttled from pF and pHN into pcDNA4/TO (Zeocin) and pcDNA5/TO vectors (Hygromycin, Invitrogen), respectively for tetracycline-regulated expression to yield pF/TO and pHN/TO. Both plasmids are transfected into B241TR by CaPO₄ coprecipitation. Single colonies are selected from transfected cells in selection media containing both Zeocin and Hygromycin. Cell clones that form syncytia (due to F and HN expression) upon tetracycline induction are chosen for further analyses. Cells with high expression of F and HN without induction are negatively selected out due to syncytium formation and cell death. With the remaining clones, western blot analyses of total cell lysates are performed with anti-F and HN antibodies, before and after induction by tetracycline. Cells that exhibit a high level of inducible expression of F and HN are tested for producing pseudotyped EIAV vector by transient transfection with pUNC-GFP. Clones that yield high titers of packaged viruses are selected as the packaging cell lines (FHN-TR).

To produce PIV3 pseudotyped EIAV vectors using the packaging cells developed above, FHN-TR are transfected with either UNC-GFP or UNC-CFTR in the presence of 10 mM sodium butyrate. Virus-containing media is harvested at 48 hrs after transfection and titrated as described above. Viral supernatants are concentrated at 50,000.g for 2 hrs. The pellet is resuspended in Hank's balanced salt solution with 1 mM MgCl₂ and 1 mM CaCl₂. The percentage of total infectivity recovered after ultracentrifugation is calculated.

The ultrastructure of the pseudotyped virus is examined by transmission electron microscopy (EM). For comparison, VSV-G pseudotyped EIAV (Olsen (1998) Gene Ther. 5:1481-1487) is also included for EM studies. Viruses are produced by the transient four-plasmid transfection or by using the packaging cell line as describe above. At 48 hrs post transfection, cells are fixed in 4% PFA, postfixed with 1% osmium tetroxide, enclosed in 1% agar, treated with 1% uranyl acetate, and embedded in Epon. Ultrathin-sections (90 nm) are analyzed with a Zeiss EM900 transmission electron microscope. To immunolabel viruses with PIV3 HN specific mAbs, transfected 293 cells are washed, fixed with 4% PFA, and incubated with antibody against PIV3 HN and 15=m gold-conjugated secondary antibody. At the end of incubation, cells are washed, pelleted, refixed with 2.5% glutaraldehyde, and processed for transmission EM as described above.

To test for replication-competent virus contamination during viral production, marker rescue assays are performed as described previously (Olsen (1998) Gene Ther. 5:1481-1487). 293 cells are transduced with PIV3 pseudotyped EIAV-GFP viruses generated from either the four-plasmid cotransfection of 293 cells or from the packaging cells. Transduced cells are cultured for 1 week, after which conditioned media is harvested and used to transduce naïve 293 cells in the presence of 8 μg/ml polybrene. 72 hrs post-transduction, the cells are scored for GFP expression. Vector stock is considered helper free if no green cells are observed.

### EXAMPLE 7

### Assessment of the Efficacy of Gene Transfer by PIV3 Pseudotyped EIAV Vectors in CF RAE Cultures

The efficiency of PIV3 pseudotyped EIAV-CFTR vector in correcting the bioelectric defect in CF HAE cultures is assessed as for PIV3CFTR viruses using identical protocols as described in Example 5. Since EIAV vector is replication defective, higher doses of viral vectors may be necessary to achieve ion transport correction.

The highest achievable dose and serial dilutions are used for gene transfer experiments. The pseudotyped EIAV vector expressing GFP is inoculated onto both the apical and basolateral surfaces of HAE cultures, and GFP expression is assessed to determine the polarity and efficiency of PIV3 pseudotyped EIAV transduction. EIAV expressing CFTR is used to transduce CF HAE cultures from the apical surface, and the efficiency of CFTR gene transfer by the pseudotyped virus is determined by bioelectrical measurement as described for PIV3CFTR as described in Example 5.

The foregoing is illustrative of the present invention, and is not to be construed as limiting thereof. The invention is defined by the following claims, with equivalents of the claims to be included therein.

1. An infectious recombinant viral vector comprising a viral genome comprising a heterologous nucleic acid of interest, wherein the viral vector is selected from the group consisting of a parainfluenza virus (PIV) and a respiratory syncytial virus (RSV) vector.

2. The vector of claim 1, wherein the vector is attenuated.

3. The vector of claim 1, wherein the vector is a human PIV vector.

4. The vector of claim 1, wherein the vector is a human RSV vector.

5. The vector of claim 3, wherein the human PIV vector is selected from the group consisting of a human parainfluenza virus-1 (PIV1) vector, a human parainfluenza virus-2 (PIV2) vector, a human parainfluenza virus-3 (PIV3) vector, and a human parainfluenza virus-4 (PIV4) vector.

6. The vector of claim 5, wherein the vector is a human PIV3 vector.

7. The vector of claim 3, wherein the viral genome comprises a regulatory element comprising an extragenic 3′ leader region and a 5′ trailer region wherein the 3′ leader region comprises a promoter wherein transcription is initiated at the 3′ leader region.

8. The vector of claim 3, wherein the nucleic acid of interest is flanked by PIV transcription and termination signals, wherein the PIV transcription and termination signals direct expression of the nucleic acid of interest.

9. The vector of claim 1, wherein the nucleic acid of interest is inserted downstream from a PIV 3′ promoter.

10. The vector of claim 3, wherein the nucleic acid of interest is inserted into a downstream non-coding region of a PIV gene selected from the group consisting of an NP, P/C/D/V, M, F, HN, and L gene.

11. The vector of claim 10, wherein the nucleic acid of interest is inserted proximal to the promoter into the downstream non-coding region of the viral genome.

12. The vector of claim 10, wherein the nucleic acid is inserted between the NP and P coding regions of the PIV genome.

13. The vector of claim 10, wherein the nucleic acid is inserted between the P and M coding regions of the PIV genome.

14. The vector of claim 10, wherein the nucleic acid is inserted between the M and HN coding regions of the PIV genome.

15. The vector of claim 10, wherein the nucleic acid is inserted between the HN and L coding regions of the PIV genome.

16. The vector of claim 10, wherein the nucleic acid is inserted upstream from the 5′ trailer region and downstream from the L coding regions of the PIV genome.

17. The vector of claim 5, wherein a translational start site of the nucleic acid of interest is preceded upstream by a SacII site.

18. The vector of claim 5, wherein an ATG translational start site is placed upstream of the translational start site of the nucleic acid of interest and in a different reading frame from the translational start site of the nucleic acid of interest.

19. The vector of claim 18, wherein expression of the nucleic acid of interest is reduced.

20. The vector of claim 3, wherein the nucleic acid of interest encodes a protein or peptide.

21. The vector of claim 3, wherein the nucleic acid of interest encodes a protein or peptide selected from the group consisting of cystic fibrosis transmembrane conductance regulator protein (CFTR) or an active fragment thereof, α₁-antitrypsin, interleukin-10 (IL-10), erytropoietin, clotting factors, and Green Fluorescent Protein, or combinations thereof.

22. An infectious recombinant PIV vector comprising a viral genome comprising a heterologous nucleic acid of interest encoding a cystic fibrosis transmembrane conductance regulator protein (CFTR) or an active fragment thereof.

23. The vector of claim 22, wherein the nucleic acid of interest encodes a human CFTR.

24. The vector of claim 4, wherein the viral genome comprises a regulatory element comprising an extragenic 3′ leader region or 5′ trailer region comprising a promoter wherein transcription is initiated at the 3′ leader region.

25. The vector of claim 4, wherein the nucleic acid of interest is flanked by RSV initiation and termination signals, wherein the RSV initiation and termination signals direct expression of the nucleic acid of interest.

26. The vector of claim 4, wherein the nucleic acid of interest is inserted downstream from an RSV 3′ promoter.

27. The vector of claim 4, wherein the nucleic acid of interest is inserted into the downstream non-coding region of an RSV gene selected from the group consisting of a NS1, NS2, N, P, M, SH, G, F, M2 and L gene.

28. The vector of claim 27, wherein the nucleic acid of interest is inserted proximal to the promoter into the downstream non-coding region of the viral genome.

29. The vector of claim 27, wherein the nucleic acid of interest is inserted between the NS1 and NS2 coding regions of the RSV genome.

30. The vector of claim 27, wherein the nucleic acid of interest is inserted between the NS2 and N coding regions of the RSV genome.

31. The vector of claim 27, wherein the nucleic acid of interest is inserted between the N and P coding regions of the RSV genome.

32. The vector of claim 27, wherein the nucleic acid of interest is inserted between the P and M coding regions of the RSV genome.

33. The vector of claim 27, wherein the nucleic acid of interest is inserted between the M and SH coding regions of the RSV genome.

34. The vector of claim 27, wherein the nucleic acid of interest is inserted between the SH and G coding regions of the RSV genome.

35. The vector of claim 27, wherein the nucleic acid of interest is inserted between the G and F coding regions of the RSV genome.

36. The vector of claim 27, wherein the nucleic acid interest is inserted between the F and M2 coding regions of the RSV genome.

37. The vector of claim 27, wherein the nucleic acid of interest is inserted upstream from the 5′ trailer region and downstream of the L coding region of the RSV genome.

38. The vector of claim 4, wherein the nucleic acid of interest encodes a protein or peptide.

39. The vector of claim 4, wherein the nucleic acid of interest encodes a protein or peptide selected from the group consisting of CFTR or an active fragment thereof, α₁-antitrypsin, IL-10, clotting factors, and erythropoietin, and Green Fluorescent Protein, or combinations thereof.

40. An infectious recombinant RSV vector comprising a viral genome comprising a heterologous nucleic acid of interest encoding a cystic fibrosis transmembrane conductance regulator protein (CFTR) or an active fragment thereof

41. The vector of claim 40, wherein the nucleic acid of interest encodes a human CFTR or active fragment thereof.

42. A composition comprising the vector of claim 1 in a physiologically acceptable carrier.

43. A method of administering the composition of claim 42, wherein the composition is administered to reach cells selected from the group consisting of lung cells, cells of the eye, epithelial cells, muscle cells, dendritic cells, pancreatic cells, hepatic cells, myocardial cells, bone cells, hematopoietic stem cells, spleen cells, keratinocytes, fibroblasts, endothelial cells, cells of the bile duct, prostate cells, cells of the vas deferens, and cells of the sweat glands/ducts.

44. A method of administering the composition of claim 42, wherein the composition is administered to the respiratory tract of a subject.

45. A method of administering the composition of claim 42, wherein the composition is administered to prevent or treat cancer or tumor of the respiratory tract of a subject.

46. The method of administering the composition according to claim 44, wherein the subject is human.

47. A method of administering the composition of claim 42, wherein the composition is administered by spray, droplet, or aerosol.

48. A pseudotyped recombinant viral vector comprising (i) a viral envelope and (ii) a viral genome comprising a heterologous nucleic acid of interest, wherein the viral envelope comprises a structural protein selected from the group consisting of:
(a) a parainfluenza virus (PIV) F and/or HN protein, and
(b) a respiratory syncytial virus (RSV) F, SH, and/or G protein.

49. The vector of claim 48, wherein the vector is attenuated.

50. The vector of claim 48, wherein the nucleic acid of interest is selected from the group consisting of CFTR or an active fragment thereof, α₁-antitrypsin, IL-10, and Green Fluorescent Protein, or combinations thereof.

51. The vector of claim 48, wherein the structural protein is a PIV F and/or HN protein.

52. The vector of claim 48, wherein the structural protein is an RSV F, SH, and/or G protein.

53. The vector of claim 48, wherein the structural protein is an RSV F and/or G protein.

54. The vector of claim 48, wherein the vector is a lentiviral vector pseudotyped with a PIV or RSV envelope protein.

55. The vector of claim 54, wherein the vector is an equine infectious anemia virus (EIAV).

56. The vector of claim 54, wherein the vector is pseudotyped with a PIV F and/or HN protein.

57. The vector of claim 54, wherein the vector is pseudotyped with PIV3 F and/or HN protein.

58. The vector of claim 54, wherein the vector is pseudotyped with an RSV F, SH, and/or G protein.

59. The vector of claim 54, wherein the vector is pseudotyped with an RSV F and/or G protein.

60. A composition comprising the vector of claim 48, in a physiologically acceptable carrier.

61. A method of administering the composition of claim 60, comprising introducing a viral vector comprising the nucleic acid of interest into a respiratory tract of a subject so that the nucleic acid of interest is expressed therein.

62. The method of administering the composition according to claim 61, wherein the subject is human.

63. The method of administering the composition of claim 60, wherein the composition is administered by spray, droplet, or aerosol.

64. A method of delivering a heterologous nucleic acid of interest into an airway epithelial cell, comprising:
introducing a viral vector comprising the nucleic acid of interest into the airway epithelial cell so that the nucleic acid of interest is expressed therein,
wherein the viral vector is a paramyxovirus virus vector selected from the group consisting of a parainfluenza virus (PIV) and a respiratory syncytial virus (RSV) vector.

65. The method according to claim 64, wherein the viral vector is attenuated.

66. The method according to claim 64, wherein the viral vector is a human respiratory syncytial virus vector.

67. The method according to claim 64, wherein the viral vector is a human parainfluenza virus vector.

68. The method according to claim 67, wherein the human parainfluenza virus vector is selected from the group consisting of a human parainfluenza virus-1 (PIV1) vector, a human parainfluenza virus-2 (PIV2) vector, a human parainfluenza virus-3 (PIV3) vector, and a human parainfluenza virus-4 (PIV4) vector.

69. The method according to claim 64, wherein the human parainfluenza virus vector is a human PIV3 vector.

70. The method according to claim 64, wherein the airway epithelial cell is a human airway epithelial cell.

71. The method according to claim 70, wherein the human airway epithelial cell is a human ciliated airway epithelial cell.

72. The method according to claim 64, wherein the introducing step is carried out in vivo.

73. The method according to claim 64, wherein the introducing step is carried out in vitro.

74. The method according to claim 64, wherein the nucleic acid of interest is operatively associated with a promoter, which promoter is active in human ciliated airway epithelial cells.

75. The method according to claim 74, wherein the nucleic acid of interest is proximal to the promoter.

76. The method according to claim 64, wherein the nucleic acid of interest encodes a protein or peptide.

77. The method according to claim 76, wherein the nucleic acid of interest encodes a protein or peptide selected from the group consisting of CFTR or an active fragment thereof, α₁-antitrypsin, IL-10, and Green Fluorescent Protein, or combinations thereof.

78. The method according to claim 77, wherein the nucleic acid of interest encodes CFTR or an active fragment thereof.

79. The method according to claim 78, wherein the nucleic acid of interest encodes a human CFTR or an active fragment thereof.

80. The method according to claim 64, wherein the introducing step is carried out by infecting the airway epithelial cell with the viral vector.

81. The method according to claim 64, wherein the airway epithelial cell is a ciliated airway epithelial cell and the viral vector is introduced from an apical surface thereof.

82. A method of delivering a heterologous nucleic acid of interest into a human ciliated airway epithelial cell, comprising:
introducing a viral vector comprising the nucleic acid of interest into the human ciliated airway epithelial cell so that the nucleic acid of interest is expressed therein,
wherein the viral vector is a PIV vector and the nucleic acid of interest encodes the CPTR protein or an active fragment thereof.

83. A method of delivering a heterologous nucleic acid of interest into a human ciliated airway epithelial cell, comprising:
introducing a viral vector comprising the nucleic acid of interest into the human ciliated airway epithelial cell so that the nucleic acid of interest is expressed therein,
wherein the viral vector is an RSV vector and the nucleic acid of interest encodes the CFTR protein or an active fragment thereof.
